(19) United States
(12) Patent Application Publication
(10) Pub. No.: US 2024/0239905 A1
(43) Pub. Date: Jul. 18, 2024

(54) A PLANT PRODUCED ANTI-EGFR MABS WITH SPECIFIC GLYCOSYLATION TO IMPROVE THE EFFICACY AGAINST CANCER

(71) Applicant: ARIZONA BOARD OF REGENTS ON BEHALF OF ARIZONA STATE UNIVERSITY, Scottsdale, AZ (US)

(72) Inventors: Qiang CHEN, Chandler, AZ (US); Ming YANG, Chandler, AZ (US); Haiyan SUN, Chandler, AZ (US); Huafang LAI, Chandler, AZ (US)

(21) Appl. No.: 18/560,207

(22) PCT Filed: May 13, 2022
(86) PCT No.: PCT/US2022/029130

§ 371 Date: Nov. 10, 2023

Related U.S. Application Data
(60) Provisional application No. 63/188,360, filed on May 13, 2021.

(51) Int. Cl. C07K 16/28; A61P 35/00; C07K 14/415; C12N 15/82

(52) U.S. Cl. CPC C07K 16/2863; A61P 35/00; C07K 14/415; C12N 15/8205; C12N 15/8257; C07K 2317/92; C07K 2319/02

(57) ABSTRACT

The present invention provides plant produced anti-EGFR antibodies that have defined glycosylation patterns. Also provided are plant expression vectors encoding said antibodies, transformed plants that express said antibodies, and methods of using said antibodies to treat cancer.

### CROSS-REFERENCE TO RELATED APPLICATIONS

This patent application claims the benefit of priority of U.S. Provisional Patent Application No. 63/188,360, filed May 13, 2021, which is incorporated herein by reference in its entirety.

### SEQUENCE LISTING

This application is being filed electronically via EFS-Web and includes an electronically 10 submitted Sequence Listing in .txt format. The .txt file contains a sequence listing entitled “112624.01330_ST25.txt” created on Apr. 14, 2022 and is 20,214 bytes in size. The Sequence Listing contained in this .txt file is part of the specification and is hereby incorporated by reference herein in its entirety.

### BACKGROUND

Colorectal cancer (CRC) is one of the most common cancers affecting both men and women, about 4.3% of the population in the United States may be diagnosed with CRC in their life time according to data collected from 2012 to 2014 (NCI, Buswell, Medina-Bolivar et al. 2005). In the past two decades, monoclonal antibodies (cetuximab and panitumumab) targeting the epidermal growth factor receptor (EGFR) have been used successfully for the treatment of metastatic CRC (Sobani, Sawant et al. 2016). However, about 40% of CRC patients who have mutations within the G protein Kras downstream of the intracellular EGFR signaling pathway do not respond to anti-EGFR therapy (Lievre, Bachet et al. 2006, Karapetis, Khambata-Ford et al. 2008, Douillard, Oliner et al. 2013) due to the constitutive activation of the intrinsic GTPase (Scheffzek, Ahmadian et al. 1997). Patients having Kras mutations were shown to have significantly shorter progression free survival and overall survival (Therkildsen, Bergmann et al. 2014). In addition to CRC, Kras mutation rates are also significant in several other types of cancer such as lung cancer or pancreatic cancer (Prior, Lewis et al. 2012). Thus, there is an urgent need to develop new therapeutics or optimize current available antibodies for treating cancers with Kras mutations.

Antibodies targeting the cancer cells act either through binding of surface antigens thereby inducing cancer cell apoptosis or by activating the antibody dependent cellular cytotoxicity (ADCC) or complement dependent cytotoxicity (CDC) pathways to kill the target cancer cells (Weiner 2015). It has been demonstrated that ADCC and CDC activities could play crucial role in antibody therapies (Weiner 2018). Therefore, many approaches have been explored to enhance ADCC or CDC activities by crystallizable fragment (Fc) glycan engineering or protein engineering. Early studies with antibody variants generated from fucosylation deficient cell lines demonstrated that removal of core fucosylation in human IgGI improved the binding affinity to Fc gamma receptor type IIIa (FcγRIIIa) and dramatically enhanced ADCC (Shields, Lai et al. 2002, Shinkawa, Nakamura et al. 2003). Later animal experiments revealed that a defucosylated chimeric antibody against CC chemokine receptor 4 had significantly higher 10 anti-tumor efficacy than that of its high fucose version (Niwa, Shoji-Hosaka et al. 2004). More importantly, the enhancement of ADCC through Fc defucosylation is independent of the FcγRIIIa phenotypes (Niwa, Hatanaka et al. 2004) indicating it could have therapeutic effect for all patients. In addition to the predominant effect of defucosylation, galactosylation or sialylation have been shown to modulate ADCC activities in the presence of fucosylation (Thomann, Reckermann et al. 2016, Li, DiLillo et al. 2017). Concurrently, Fc variants may improve complement binding and enhance CDC activity. For example, mutations in the Fc region that can promote the formation of hexamers in the presence of antigen showed potent CDC activities (Diebolder, Beurskens et al. 2014, de Jong, Beurskens et al. 2016). These approaches may be applicable for improving the cancer antibody therapeutic effects for patients with downstream mutations in the EGFR signaling pathways.

### SUMMARY

The present invention provides anti-EGFR antibodies comprising: a heavy chain comprising a polypeptide with at least 95% sequence identity to SEQ ID NO:2 (i.e., the heavy chain variable domain (VH) of cetuximab) and a light chain comprising a polypeptide with at least 95% sequence identity to SEQ ID NO:4 (i.e., the light chain variable domain (VL) of cetuximab). The antibodies are distinguished by the fact that they (a) have a glycosylation pattern selected from the group consisting of: GnGn, AA, and GnGnXF; (b) comprise an E430G mutation in the fragment crystallizable (Fc) region; or (c) both (a) and (b). In some embodiments, SEQ ID NO:2 is encoded by SEQ ID NO:1 (i.e., a codon optimized DNA 30 sequence encoding the VH of cetuximab). In some embodiments, SEQ ID NO:4 is encoded by SEQ ID NO:3 (i.e., a codon optimized DNA sequence encoding the VL of cetuximab).

In a second aspect, the present invention provides plant expression vectors encoding an anti-EGFR antibody comprising: a heavy chain comprising SEQ ID NO:2 (i.e., the VH of cetuximab) and a light chain comprising SEQ ID NO:4 (i.e., the VL of cetuximab). The plant expression vectors may include a plant promoter operably connected to the polynucleotides encoding SEQ ID NO: 2 and SEQ ID NO: 4.

In a third aspect, the present invention provides plants that have been transformed with the plant expression vectors described herein.

In a fourth aspect, the present invention provides anti-EGFR antibody produced by the plants described herein.

In a fifth aspect, the present invention provides methods of treating cancer in a subject by administering the antibodies described herein.

### BRIEF DESCRIPTION OF THE DRAWINGS

FIG. 1 shows a western blot analysis of plant-produced anti-EGFR antibodies. Wt-Anti-EGFR and ΔXF-Anti-EGFR were extracted from wild type and AXFT N. benthamiana leaves, respectively. They were separated on SDS-PAGE gels under non-reducing (A: lanes 2 and 3) or reducing (B: lanes 2 and 3; or C: lanes 2 and 3) conditions and blotted onto PVDF membranes. Lane 1 in A, B, and C is F10, an IgG isotype used as a reference standard. The membranes were incubated with a goat anti-human kappa chain antibody to detect assembled monoclonal antibody (A) or light chain (C). A goat anti-human gamma chain antibody was used to detect heavy chain (B). Hc, heavy chain; Lc, light chain.

FIG. 2 shows the purification of wt-Anti-EGFR and ΔXF-Anti-EGFR from N. benthamiana leaves. Total soluble proteins were extracted on day 7 after agroinfiltration, then the antibodies were purified and analyzed on a 4-20% gradient SDS-PAGE gel under reducing (A) or non-reducing (B) conditions and visualized with Coomassie stain. Lane 1, F10 antibody used as an IgG isotype reference standard; lane 2, pAnti-EGFR; lane 3, pAXF-Anti-EGFR. Hc, heavy chain; Lc, light chain. One representative of several independent experiments is shown.

FIG. 3 demonstrates that plant-produced Anti-EGFR antibodies bind to CRC cell lines equivalently as CHO-expressed Anti-EGFR. Cancer cells (A. HT29, B. HCT116, C. LS174T) were first stained with 1: human IgG isotype negative control, 2: CHO-expressed Anti-EGFR mAb1, 3: CHO-expressed EGFR mAb2, and 4: plant-produced EGFR mAb (GnGn glycans).

The two CHO-cell expressed mAbs in 2 and 3 are from two different commercial sources. (FACS plots from left-most column (1) to right (4)). Binding intensity was determined by flow cytometry. Representative histograms from three independent experiments are shown.

FIG. 4 demonstrates that plant produced Anti-EGFR antibodies recognize the same epitope as cetuximab. EGFR-expressing CRC cell lines (A: HT29, B: HCT116) were first incubated with serial dilutions of unlabeled wt-Anti-EGFR (0.01-10 μg/ml) then washed twice with 1×PBS before stained with Alex488-conjugated cetuximab and processed by flow cytometry. Representative histograms from three independent experiments of each concentration of wt-Anti-EGFR were overlaid.

FIG. 5 shows an SPR analysis of binding kinetics and affinity of plant produced Anti-EGFR binding to FcγRIIIa (CD16A). Recombinant human CD16A ectodomain was injected over the surface with anti-EGFR monoclonal antibody variants captured on immobilized protein A. Referenced and blanked sensograms were fitted with BIAcore Evaluation Software using Two State Reaction model.

FIG. 6 demonstrates that plant produced Anti-EGFR antibodies enhanced NK cell ADCC activity for both wild type and Kras mutant cancer cell lines. Cancer cells (A. Caco-2, B. HCT116) were first incubated with plant produced antibodies, human IgG (negative control), or cetuximab (positive control), before added to NK cells for lysis. Lysis rate was measured and calculated as described in Methods. Representative data from three independent experiments are shown.

FIG. 7 demonstrates that plant-produced Anti-EGFR antibodies exhibit enhanced CDC activity in A431 cells. A431 cells were incubated with different antibodies (20 μg/ml) and 25% final concentration of normal human serum (NHS). The relative apoptosis fold was measured and calculated as described in Methods, each data point is the average of triplicates.

FIG. 8 shows the structures of the various glycosylation abbreviations used herein.

### DETAILED DESCRIPTION

The present invention provides plant produced anti-EGFR antibodies that have defined glycosylation patterns. Also provided are plant expression vectors encoding said antibodies, transformed plants that express said antibodies, and methods of using said antibodies to treat cancer.

In the present application, the inventors describe the expression, purification, and binding and functional characterization of a series of anti-EGFR antibodies produced in wild type, AXF and AA N. benthamiana plants. Compared to their CHO expressed counterpart, cetuximab, these plant-produced anti-EGFR antibodies preserved equivalent binding specificity and affinity to EGFR expressing CRC cells. They displayed more uniformed mammalian N-glycosylation of the GnGn and AA glycoforms, thereby improved FcγRIIIa binding and enhanced ADCC activity.

Furthermore, antibodies produced in ΔXF and AA plants or with the E430G mutation in the Fc region increased C1q binding and CDC activity. These antibodies may provide new therapeutic options for cancer patients with downstream mutations in the EGFR signaling pathway through improved ADCC and CDC activity.

Protein-based biologics have revolutionized the treatment of many diseases, but low efficacy, occasional undesirable side effects and rapid clearance from circulation limit their full potential. The majority of pharmaceutically relevant proteins are N-linked glycosylated, and their sugar moieties have significant impact on their folding, assembly, solubility, serum and shelf half-life, and functionality. Protein-associated sugars have been shown to play crucial roles in all domains of life including recognition, signaling, and adhesion within and between cells. Thus, one approach to enhance the potency, safety and stability of therapeutic proteins is glycoengineering, altering protein-associated carbohydrates to achieve the desirable protein properties. The difficult challenge is to develop biological systems that can consistently produce glycoproteins with homogeneous glycans on demand. The availability of such systems will lead to breakthroughs on two fronts: (1) elucidating the contribution of sugar moieties for various biological functions and (2) developing novel biologics with tailor-made glycosylation based on their functional needs.

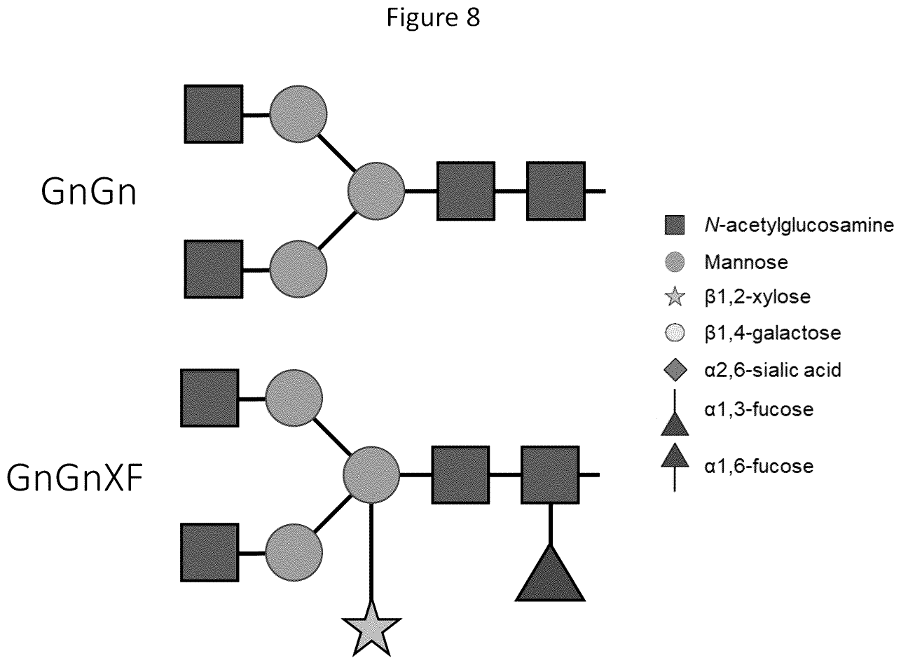

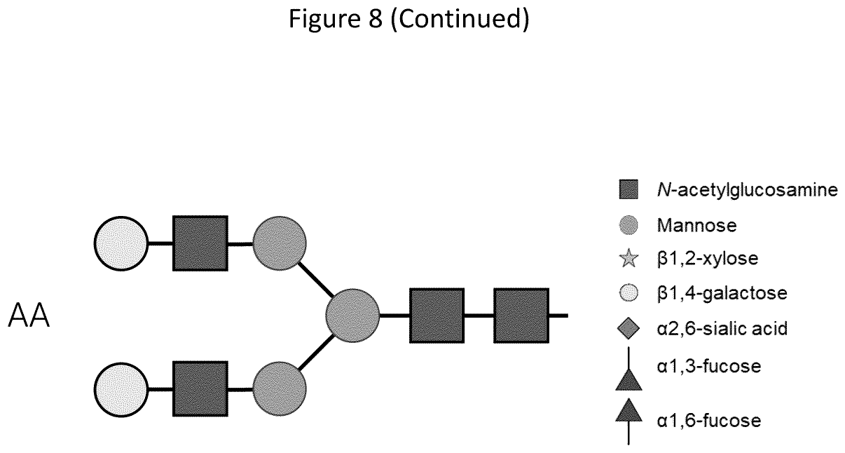

Unlike the protein backbone, which is synthesized based on a defined template, carbohydrate chains are assembled enzymatically. As a result, they are diverse in both the number and the linkage patterns of the sugar units. In addition, different host cells may modify the same protein with different sugar structures. Protein N-linked glycosylation involves the addition of an oligosaccharide (Man9) to the amino group of an asparagine residue on a nascent polypeptide. In all eukaryotes, the transfer and initial processing of Man9 starts in the endoplasmic reticulum to form Man8, which is further processed in cis and medial Golgi compartments to form complex N-glycans. The early steps of N-glycan processing are well preserved among most eukaryotic cells, especially up to the formation of the intermediate of GnGn. The GnGn structure in this application is shown in FIG. 8 with reside Asn297 the site of cetuximab glycosylation. However, processing beyond this point differs significantly between eukaryotes, leading to the formation of different complex N-glycoforms. In mammalian cells, there is a large diverse population of glycoenzymes for extensive elongation of the GnGn substrate; these give rise to more than 2000 different N-glycans generated by a few hundred enzymes in the secretory pathway. The majority of large pharmaceutical companies currently use Chinese hamster ovary (CHO) cells to produce human biologics. CHO cells generally produce N-glycans with branching and capping similar to their human counterparts. Due to its large glycome, however, CHO cell-derived glycoproteins exhibit substantial glycan heterogeneity, precluding the ability to generate distinct glycoforms that could be used in comparative studies of specific biological effects. Furthermore, N-glycan processing in CHO cells is prone to environmental variation and is difficult to control during bioprocessing. This has led to CHO cell glycoengineering efforts to control their glycosylation capacity and reduce heterogeneity.

However, the overall success of glycoengineering in CHO cells has been relatively modest, especially in producing defined N-glycoforms with high degrees of homogeneity. This and the lack of reported success by chemical synthesis have encouraged the development of alternative expression systems that can produce distinct human glycoforms on demand.

Plants have been explored as an alternative platform for producing protein biologics with the expectation that they will decrease protein production costs and give high scalability and increased safety. The most exciting aspect of plant-based systems for biologic development is their amenability for glycoengineering. In contrast to mammals, plant cells have a drastically reduced repertoire of Golgi-located glycoenzymes, and give rise to only two dominant glycan structures, GnGnXF and MMXF. As a result, unlike mammalian cell-derived recombinant proteins that carry a mixture of several N-glycans, plant produced proteins usually bear a single dominant N-glycan structure. The two major plant N-glycoforms contain core α1,3-fucose and 01,2-xylose which are not present in glycoproteins produced by human cells. Concerns were raised that biologic proteins produced in plants might trigger immune responses leading to production of plant-glycan specific antibodies that could reduce their efficacy or cause adverse effects. Paradoxically, the limited repertoire of glycoenzymes for N-glycosylation has turned out to be an advantage for plant cells as a host for generating proteins with homogeneous glycans, in contrast to the large glycome and the resulting glycan heterogeneity that impedes the targeted manipulation of the N-glycosylation pathway in mammalian cells. Plants exhibit a remarkable tolerance toward various glycan manipulations and display no major phenotypic changes in growth or development in response to deletion, insertion and substitution of their native glycan structures. The general approach for plant glycoengineering is to first delete or suppress the expression of glycoenzymes for synthesizing non-human sugars and subsequently, build human glycoforms by introducing mammalian glycoenzymes. Consequently, a Nicotiana benthamiana line called ΔXF that does not produce the plant-specific α1,3-fucose and β1,2-xylose was created by suppressing the expression of two plant-specific glycantransferases. The successful development of ΔXF plants not only eliminates the concern for the immunogenicity of plant-produced glycoproteins, but also demonstrates the plasticity of plants in tolerating the manipulation of their native glycosylation pathway. Moreover, various monoclonal antibodies (mAbs) produced in ΔXF plants have been shown to carry a homogenous (>90%) GnGn N-glycan structure compared to the 5-7 glycan structures exhibited by the same mAbs produced in CHO cells. Functional analysis revealed that they have increased neutralization activity and/or significantly enhanced ADCC potency. These enhancements were highlighted by ZMapp, a cocktail of three anti-Ebola mAbs produced in ΔXF plants. They have a superior potency to their CHO cell-produced counterparts and were able to rescue 100% of rhesus macaques even when given 5 days after a lethal Ebola challenge, leading to ZMapp's compassionate use in human patients during the 2014 Ebola outbreak.

The ΔXF plants also provide GnGn, a vital glyco-substrate for further humanization of the N-glycosylation pathway. Subsequently, a series of successes were achieved in N. benthamiana plants in producing various defined human N-glycan structures including α1,6 core fucose, bisected and tetra-antennary complex N-glycans, and bigalactosylated N-glycoforms.

Glycoengineered ΔXF plants can now produce mAbs with identical N-glycosylation profiles, differing only in core α1,6-fucose, overcoming CHO cells' inability to synthesize multiantennary N-glycans, and providing an optimal substrate for terminal sialylation. These studies also revealed that fine-tuning the sub-organelle localization of the introduced glycoenzymes is crucial for producing the target human glycoforms as random introduction of mammalian enzymes would interfere with the endogenous glycosylation pathway and produce incomplete or unusual hybrid N-glycans. This knowledge has led to the success of producing bi-antennary α2,6-sialylated N-glycans by the simultaneous expression and precise targeting of six mammalian glycoenzymes to various subcellular compartments using a transient expression system.

Antibodies with homogenous N-glycans offer several advantages. First, the N-linked sugar moieties on antibodies have significant impact on their folding, assembly, solubility, serum and shelf half-life, and functionality. Second, the specific functions of glycans include affecting the binding dynamics and binding affinity of antibody to various Fc receptors, leading to either beneficial effector functions (such as antibody-dependent cell cytotoxicity (ADCC), antibody dependent phagocytosis (ADPC), and complement dependent cytotoxicity (CDC) to clear pathogens) or adverse effect (such as antibody dependent enhancement of infection). Third, when a mAb carries a mixture of various glycans, it would not be very efficacious or safe, because only a fraction of the mAb would carry the effective glycan(s) to give efficacy. mAb population that carries other glycans in the mixture may not have any efficacy at all, or even worse, may cause serious side effects. Fourth, the ability of producing mAbs with homogenous glycans on demand allow us to (1) select the most efficacious and safest mAb glycoform (when you can only obtain a mAb prep with a mixture of glycans, you would not be able to tell which type of glycan is responsible for efficacy or side effects) and (2) to produce mAb with the glycans selected in (1) with high homogeneity. This way, you are sure your antibody drug is free of the glycans that cause side effects, and the majority of the mAbs in your drug have the best possible efficacy.

Antibodies: In a first aspect, the present invention provides anti-EGFR antibodies comprising: a heavy chain comprising a polypeptide with at least 95% sequence identity to SEQ ID NO:2 (i.e., the heavy chain variable domain (VH) of cetuximab) and a light chain comprising a polypeptide with at least 95% sequence identity to SEQ ID NO:4 (i.e., the light chain variable domain (VL) of cetuximab). The antibodies are distinguished by the fact that they (a) have a glycosylation patterns selected from the group consisting of: GnGn, AA, and GnGnXF; (b) comprise an E430G mutation in the fragment crystallizable (Fc) region; or (c) both (a) and (b). In some embodiments, SEQ ID NO:2 is encoded by SEQ ID NO:1 (i.e., a codon optimized DNA sequence encoding the VH of cetuximab). In some embodiments, SEQ ID NO:4 is encoded by SEQ ID NO:3 (i.e., a Nicotiana codon optimized DNA sequence encoding the VL of cetuximab).

The term “antibody” refers to immunoglobulin molecules or other molecules that comprise an antigen-binding domain from an immunoglobulin molecule. Suitable antibody molecules include, without limitation, whole antibodies (e.g., IgG, IgA, IgE, IgM, or IgD), monoclonal antibodies, polyclonal antibodies, chimeric antibodies, humanized antibodies, and antibody fragments, and genetically engineered antibodies. Thus, any form of antibody, antibody fragment, or antibody-derived fragment may be used with the present invention, as long as it retains the ability to bind to EGFR in vivo.

Whole antibodies comprise at least two heavy (H) chains and two light (L) chains. Each heavy chain is comprised of a heavy chain variable region (VH) and a heavy chain constant region. The heavy chain constant region is comprised of three domains, CH1, CH2 and CH3. Each light chain is comprised of a light chain variable region (VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen (e.g., EGFR). The fragment crystallizable region (Fc region) is the tail region of an antibody, which comprises the CH2 and CH3 domains of the heavy chain constant region. The Fc region interacts with cell surface receptors called Fc receptors and some proteins of the complement system.

As stated above, the antibodies of the present invention may be antibody fragments. However, the antibodies must have a specific glycosylation pattern at a glycosylation site in the CH2 domain of the Fc region and/or comprise an E430G mutation in the Fc region. As the inventors have demonstrated, antibodies with these modifications provide the enhanced ADCC and CDC activity. Thus, the antibody fragments used with the present invention generally comprise the Fc region. Suitable antibody fragments include Fc-fusion proteins such as a single-chain variable fragment (scFc)-Fc region fusion protein or a diabody-Fc region fusion protein.

Other suitable antibody fragments that may be included in the Fc-fusion proteins include, for example, Fab, Fab′, F(ab′)2, Fv, dsFv, ds-scFv, Fd, dAbs, TandAbs dimers, mini bodies, monobodies, and bispecific antibody fragments. A fragment is suitable for use in the present methods and kits if it retains the ability to bind in vivo to EGFR.

Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Protein and nucleic acid sequence identities are evaluated using the Basic Local Alignment Search Tool (“BLAST”), which is well known in the art (Karlin and Altschul, 1990, Proc. Natl. Acad. Sci. USA 87: 2267-2268; Altschul et al., 1997, Nucl. Acids Res. 25: 3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990), the disclosure of which is incorporated by reference in its entirety. The BLAST programs can be used with the default parameters or with modified parameters provided by the user.

In some embodiments, the heavy chain comprises a polypeptide with at least 85%, at least 86%, at least 87%, at least 88%, at least 89%, at least 89%, at least 90%, at least 91%, at least 92%, at least 93%, at least 94%, at least 95%, at least 96%, at least 97%, at least 98%, at least 99% or more sequence identity to SEQ ID NO:2. In some embodiments, the light chain comprises a polypeptide with at least 85%, at least 86%, at least 87%, at least 88%, at least 89%, at least 89%, at least 90%, at least 91%, at least 92%, at least 93%, at least 94%, at least 95%, at least 96%, at least 97%, at least 98%, at least 99% or more sequence identity to SEQ ID NO:4.

Cetuximab (Erbitux®) is a recombinant chimeric monoclonal antibody that binds to the extracellular domain of the human epidermal growth factor receptor (EGFR) and is used in the clinic to treat certain forms of cancer. Unfortunately, some patients have experienced an allergic response, in some cases anaphylactic shock and death, when treated with Erbitux®. This allergic response has been shown to be caused by IgE that recognizes the galactose-alpha-1,3-galactose glycosylation molecule present on the commercial form of cetuximab, Erbitux® (N Engl J Med (2008), 358:1109-17). Addition of galactose-alpha-1,3-galactose is thought to be a result of the mammalian (murine myeloma) cell culture system used to commercially produce Erbitux®. Production of proteins in Chinese hamster ovary cells can also result in galactose-alpha-1,3-galactose moieties on the proteins (Bosques et al., Nat. Biotechn. 28: 1153-1156, 2010). Advantageously, the antibodies of the present invention do not contain these problematic glycans.

Glycosylation is a reaction in which a carbohydrate is attached to a hydroxyl or another functional group of another molecule (e.g., a protein). The term “glycan” is used to refer to the attached polysaccharide. “N-linked glycans” are a particular class of glycan that is attached to a nitrogen of asparagine or arginine side-chains. As used herein, a “glycosylation pattern” the set of glycan structures present on a particular protein (e.g., an antibody). The antibodies of the present invention may have one of three glycosylation patterns (i.e., GnGn, AA, and GnGnXF), which are shown schematically in FIG. 8. GnGn is a mammalian core N-glycan structure with two terminal β1,2-linked GlcNAc residues GlcNAc2Man3GlcNAc2. AA is a mammalian N-glycan structure with two terminal galactose residues attached to the GnGn structure. GnGnXF is a plant-specific N-glycosylation form with β1,2-xylosylation (X) and core α1,3-fucosylation (F) attached to the structure of GnGn.

E430G is a mutation on the Fc region of the heavy chain of human IgG (PLoS Biol (2016), 14(1):e1002344). Specifically, the glutamate (E) residue at position 430 in the wild-type heavy chain is substituted by glycine (G). As a result of this substitution, antibodies that carry this heavy chain mutation form hexamers on the surface of target cells more readily than non-mutant antibodies, which enhances the CDC and ADCC activity of the antibodies. In the Examples, the inventors use this mutant either (1) as an independent approach to enhance colon cancer cell killing activity through CDC or/and ADCC by plant produced cetuximab, or (2) in combination with glycoengineering (i.e., plant produced glycosylation patterns) to further enhance ADCC and CDC-mediated cancer cell killing.

For improved expression in plant cells, the inventors introduced a plant signal peptide to the heavy chain and light chain of their antibodies. As used herein, the term “plant signal peptide” refers to a peptide that ensures proper localization and glycosylation of the antibodies. Specifically, the signal peptide ensures that the antibody is produced in endoplasmic reticulum (ER) and trafficked through the endomembrane system to be secreted at the plant cell surface. Because glycoproteins are glycosylated during endomembrane system trafficking, this process is required to achieve full N-glycosylation. Thus, in some embodiments, the heavy chain and the light chain each further comprise a plant signal peptide. In some embodiments, the plant signal peptide comprises SEQ ID NO:6.

To further improve expression in plants, the inventors codon optimized the DNA sequence encoding the cetuximab antibody for expression in Nicotiana benthamiana. The term “codon optimization” refers to a genetic engineering approach in which synonymous codon substitutions are made based on an organism's codon usage bias. Codon optimization increases translational efficiency without altering the sequence of the protein.

In some embodiments, the antibodies comprise the full-length heavy chain and light chain of cetuximab with the addition of the plant signal peptide sequence. Thus, in some embodiments, the heavy chain comprises SEQ ID NO:8 (i.e., the wild-type heavy chain) or SEQ ID NO:10 (i.e., the E430G mutant heavy chain) and the light chain comprises SEQ ID NO:12. In some embodiments, SEQ ID NO:8 is encoded by SEQ ID NO:7 (i.e., a codon optimized DNA sequence encoding the wild-type heavy chain). In some embodiments, SEQ ID NO:10 is encoded by SEQ ID NO:9 (i.e., a codon optimized DNA sequence encoding the E430G mutant heavy chain). In some embodiments, SEQ ID NO:12 is encoded by SEQ ID NO:11 (i.e., a codon optimized DNA sequence encoding the light chain).

The antibodies of the present invention may comprise additional modifications as compared to the parental antibody, cetuximab. For example, the inventors added two amino acids (i.e., alanine and serine, “AS”) between the light chain variable region (VL) and the light chain constant region (CL) to facilitate efficient protein folding. See SEQ ID NO: 12.

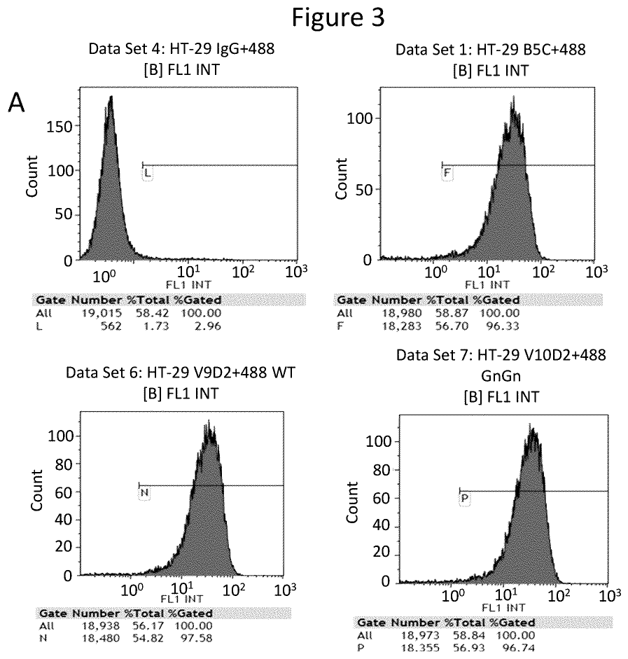

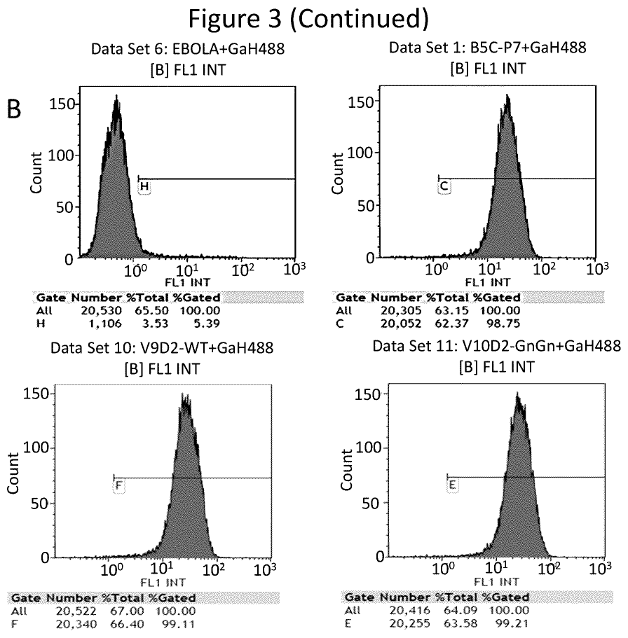

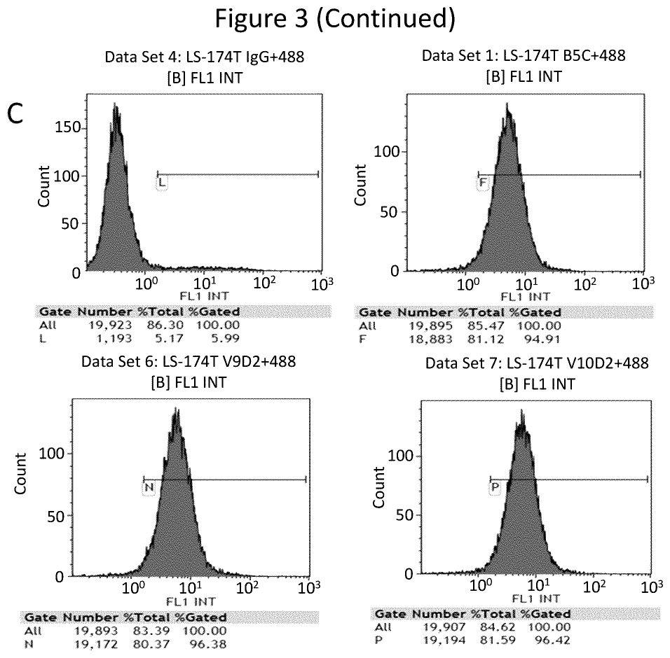

In the Examples, the inventors demonstrate that their plant produced anti-EGFR antibodies can bind to human colon cancer cell lines that express either a wild-type or a mutated K-RAS protein (see FIG. 3). Thus, in some embodiments, the antibodies bind to cancer cells that express a wild-type K-RAS protein or a mutated K-RAS protein. This suggests that the antibodies of the present invention have the potential to treat cancers that comprise K-RAS mutations, which do not respond to conventional anti-EGFR therapies (see Background).

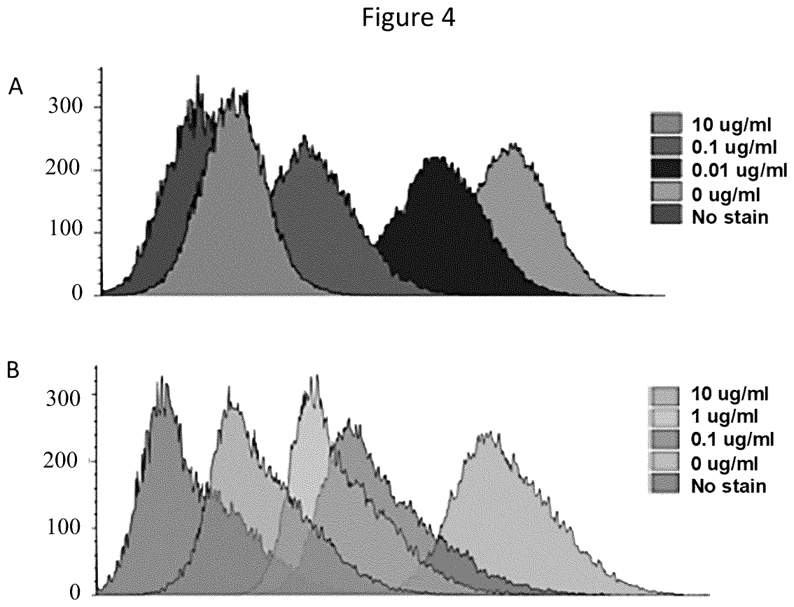

The inventors also showed that their plant produced anti-EGFR antibodies bind to the same epitope of EGFR that is bound by the commercially available antibody cetuximab (see FIG. 4). As used herein, the term “epitope” refers to the portion of an antigen that an antibody specifically binds to. Thus, in some embodiments, the antibodies bind to the same epitope of EGFR as cetuximab.

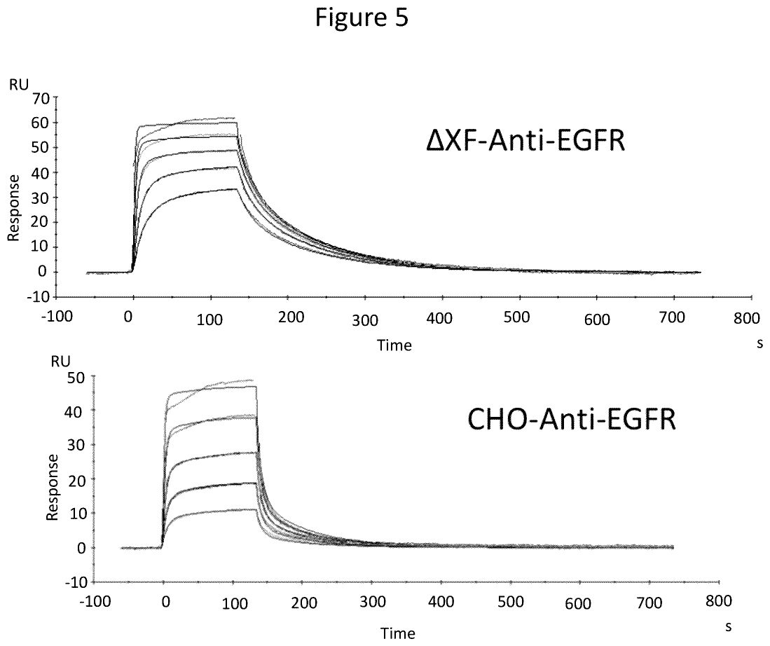

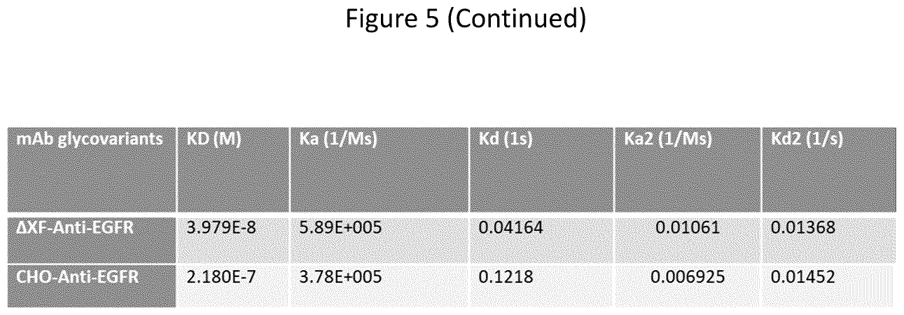

Further, the inventors demonstrated that their plant produced anti-EGFR antibodies have a higher affinity to cluster of differentiation 16A (CD16A, also referred to as FcγRIIIA) and to complement component 1q (C1q) than cetuximab (see FIG. 5). As used herein, the term “affinity” refers to the binding energy between an antibody and an antigen. CD16A is an activating receptor that is mostly expressed on natural killer (NK) cells and monocytes/macrophages. It mediates antibody-dependent cell-mediated cytotoxicity (ADCC) through low-affinity interaction with human immunoglobulin G (IgG) Fc. C1q is a protein complex involved in the complement system. Binding of antigen-antibody complexes to C1q activates the C1 complex and initiates the classical complement pathway of the complement system. Thus, in some embodiments, the antibodies have a higher affinity to CD16A than cetuximab. In some embodiments, the antibodies have a higher affinity to C1q than cetuximab.

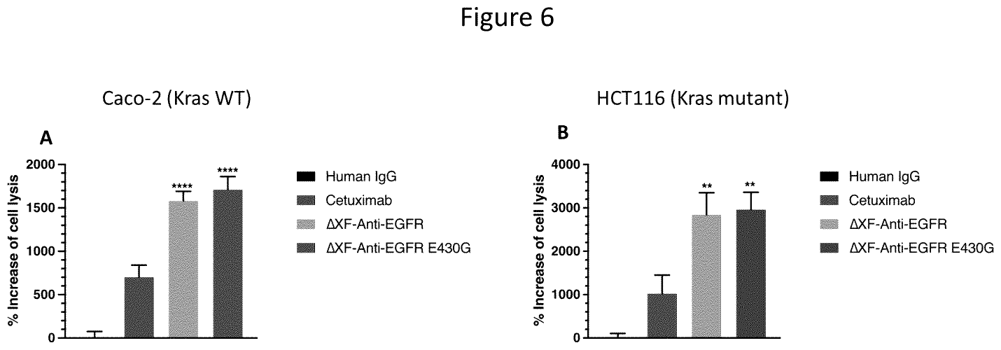

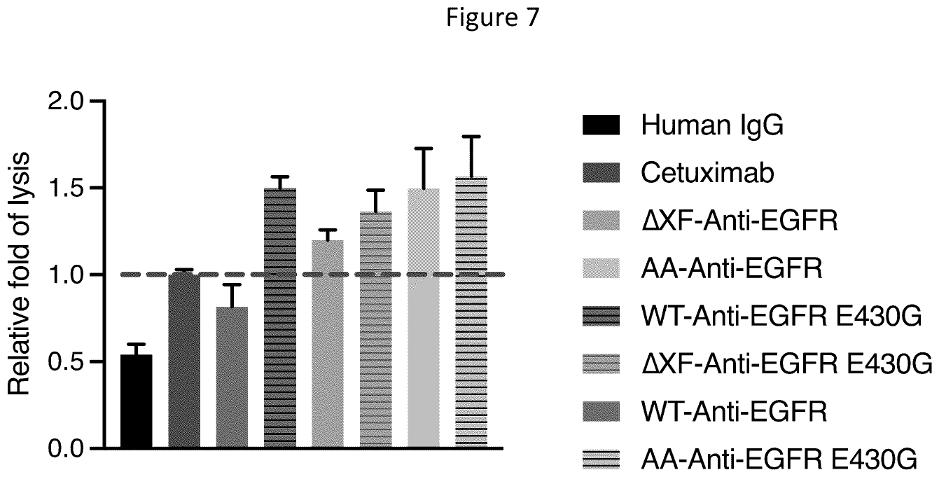

Finally, the inventors demonstrated that their plant produced anti-EGFR antibodies may be more effective for killing cancer cells as compared to cetuximab. Antibodies can kill cancer cells by several mechanisms: (1) some bind to surface antigens and induce cancer cell apoptosis, (2) some activate the antibody dependent cellular cytotoxicity (ADCC), and (3) some activate complement dependent cytotoxicity (CDC). In ADCC, antibodies recruit effector cell (e.g., natural killer cells) that lyse the target cell. In CDC, antibodies bind to the protein C1q, leading to the formation of a membrane attack complex (MAC) and the activation of the complement pathway. In the Examples, the inventors show that a subset of their plant produced antibodies (i.e., ΔXF-Anti-EGFR and ΔXF-Anti-EGFR E430G) have higher cancer cell lysis rates than cetuximab in an ADCC assay (see FIG. 6). Importantly, enhanced killing of cancer cells via natural killer cell-mediated ADCC may provide an alternative mechanism of treatment for patients with downstream mutations in the EGFR signaling pathway, such as K-RAS mutations. Further, the inventors show that all of their plant produced antibodies exhibited higher CDC activity as compared to cetuximab, and that the E430G mutation further increases this activity (see FIG. 7). Thus, in some embodiments, the antibodies have a higher ADCC activity than cetuximab. In some embodiments, the antibodies have a higher CDC activity than cetuximab.

In some embodiments, the antibodies of the present invention comprise the full-length heavy chain and light chain of cetuximab with the addition of the plant signal peptide sequence, and they have a plant-specific glycosylation pattern. Specifically, in some embodiments, the heavy chain comprises SEQ ID NO:8 (i.e., the wild-type heavy chain sequence) or SEQ ID NO:10 (i.e., the E430G mutant heavy chain sequence) and the light chain comprises SEQ ID NO:12, and the antibodies have glycosylation patterns selected from the group consisting of: GnGn, AA, and GnGnXF.

### Plant Expression Vectors:

In a second aspect, the present invention provides plant expression vectors encoding an anti-EGFR antibody comprising: a heavy chain comprising SEQ ID NO:2 (i.e., the VH of cetuximab) and a light chain comprising SEQ ID NO:4 (i.e., the VL of cetuximab).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. “Expression vectors” are vectors that are capable of directing the expression of nucleic acids to which they are operatively linked. “Plant expression vectors” are vectors that have been specifically designed to produce protein in transgenic plants. Vectors suitable for use with the present invention comprise the nucleotide sequence encoding the antibodies described herein and a heterogeneous sequence necessary for proper propagation of the vector and expression of the encoded polypeptide. The heterogeneous sequence (i.e., sequence from a difference species than the polypeptide) can comprise a heterologous promoter or heterologous transcriptional regulatory region that allows for expression of the polypeptide. As used herein, the terms “heterologous promoter,” “promoter,” “promoter region,” or “promoter sequence” refer generally to transcriptional regulatory regions of a gene, which may be found at the 5′ or 3′ side of the vectors described herein, or within the coding region of the vectors, or within introns in the vectors. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding domains (consensus sequences) responsible for the binding of RNA polymerase.

In some embodiments, the expression vector backbone is MagnICON®. The inventors selected this vector for antibody production because it drives high-level expression quickly, typically reaching peak expression about 7-8 days after gene introduction. In other embodiments, the expression vector backbone is the bean yellow dwarf virus derived geminiviral vector (Hum Vaccin (2011), 7(3):331-8), which the reaches peak expression even faster (i.e., about 4 days after gene introduction).

In some embodiments, the heavy chain and the light chain encoded by the plant expression vector each further comprise a plant signal peptide. In some embodiments, the plant signal peptide comprises SEQ ID NO:6.

In some embodiments, the antibodies encoded by the plant expression vector comprise the full-length heavy chain and light chain of cetuximab with the addition of the plant signal peptide sequence. Thus, in some embodiments, the heavy chain comprises SEQ ID NO:8 (i.e., the wild-type heavy chain sequence) or SEQ ID NO:10 (i.e., the E430G mutant heavy chain sequence) and the light chain comprises SEQ ID NO: 12.

### Plants:

In a third aspect, the present invention provides plants that have been transformed with the plant expression vectors described herein. Advantageously, plants produce glycoproteins with defined and homogenous N-glycans.

As used herein, the term “plant” includes whole plants, plant organs (e.g., leaves, stems, flowers, roots, etc.), plant protoplasts, seeds and plant cells and progeny of same. Suitable plants include both dicotyledonous and monocotyledonous plants. In some embodiments, the plant is Nicotiana benthamiana. Importantly, in contrast to proteins expressed in mammalian expression systems, proteins expressed in wild-type N. benthamiana have only two dominant glycoforms (GnGnXF and MMXF). Other suitable plants for use with the present invention include, for example, other Nicotiana species (e.g., N. tabacum), lettuce, spinach, rice, corn, and Arabidopsis thaliana.

A “glycoform” is an isoform of a protein that differs only with respect to the number or type of attached glycans. As used herein, the term “dominant glycoform” refers to a glycoform that makes up a substantial portion of the glycoforms of a protein. For example, a dominant glycan may make up more than 10%, more than 20%, more than 25%, or more than 30% of the total amount of the species of a protein. A quantitative determination of glycans may be made, for example, by LC-MS analysis in which the area under the curve (AUC) of the peaks is compared to determine the relative quantity of each glycan in a protein batch.

In addition to expressing the antibodies in wild-type N. benthamiana, the inventors also expressed the antibodies in transgenic ΔXF and AA N. benthamiana plants, which produce proteins that only contain one dominant glycoform (i.e., GnGn in ΔXF plants, and AA in AA plants). ΔXF is a Nicotiana benthamiana line that does not produce the plant-specific glycans α1,3-fucose and β1,2-xylose, eliminating concerns regarding the immunogenicity of the plant-produced glycoproteins. This line was created by suppressing the expression of two plant-specific glycantransferase genes: the endogenous β1,2-xylosyltransferase (XylT) and α1,3-fucosyltransferase (FucT) genes (Plant Biotechnology Journal (2008), 6(4):392-402). AA (alternative name: ^{ST}GalT-ΔXF) is a Nicotiana benthamiana line that stably expresses a β1,4-galactosyltransferase (^{ST}GalT) in the ΔXF plant background. AA produces antibodies with the AA N-glycosylation pattern (Plant Physiol Biochem (2015), 92:39-47). Thus, in some embodiments, the plant is a ΔXF plant or an AA plant. In some embodiments, the plant expresses 1-2 dominant glycoforms of the antibody encoded by the plant expression vector.

The plants of the present invention were generated to produce the anti-EGFR antibodies described herein. Accordingly, in a fourth aspect, the present invention provides anti-EGFR antibody produced by the plants described herein. In some embodiments, the antibodies have a glycosylation pattern selected from the group consisting of: GnGn, AA, and GnGnXF.

### Methods

In a fifth aspect, the present invention provides methods of treating cancer in a subject by administering the antibodies described herein.

As used herein, “treating” or “treatment” describes the management and care of a subject for the purpose of combating a disease, condition, or disorder. Treating includes administering a treatment to prevent the onset of the symptoms or complications, to alleviate the symptoms or complications, or to eliminate the disease, condition, or disorder. For example, treating cancer in a subject includes the reducing, repressing, delaying or preventing of cancer growth, reduction of tumor volume, and/or preventing, repressing, delaying or reducing metastasis of the tumor. Treating cancer in a subject also includes the reduction of the number of tumor cells within the subject.

As used herein, “subject” or “patient” refers to mammals and non-mammals. A “mammal” may be any member of the class Mammalia including, but not limited to, humans, non-human primates (e.g., chimpanzees, other apes, and monkey species), farm animals (e.g., cattle, horses, sheep, goats, and swine), domestic animals (e.g., rabbits, dogs, and cats), or laboratory animals including rodents (e.g., rats, mice, and guinea pigs). Examples of non-mammals include, but are not limited to, birds, and the like. The term “subject” does not denote a particular age or sex. In one specific embodiment, a subject is a mammal, preferably a human.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent.

As used herein the term “cancer” refers to an abnormal mass of tissue in which the growth of the mass surpasses and is not coordinated with the growth of normal tissue. In the case of hematological cancers, this includes a volume of blood or other bodily fluid containing cancerous cells. A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites. Accordingly, a cancer cell is a cell found within the abnormal mass of tissue whose growth is not coordinated with the growth of normal tissue.

The methods of the present invention can be used to treat any cancers that express EGFR on the cancer cell surface, including, but not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma. In some embodiments, the cancer is selected from colorectal cancer, non-small cell lung cancer, and head/neck cancer.

In some embodiments, the cancer comprises a mutation in KRAS or BRAF. BRAF is a protein kinase from the RAF family, which plays an important role in the RAS/RAF signaling cascade directly downstream of Ras. V600E is the most commonly observed BRAF mutation, which has been identified in up to 7% of human cancers (Cancers (Basel) (2020), 12(6): 1571).

This mutation produces a constitutively activated protein kinase and downstream signaling, similar to that in KRAS mutant tumors. Thus, a BRAF mutation is an indicator of poor prognosis (N Engl J Med (2009), 361:98-99). Similar to patients with KRAS mutations, patients with BRAF mutations do not directly benefit from anti-EGFR antibody therapies (European Journal of Cancer (2015), 51(5): 587-594). Concomitant mutations in both KRAS and BRAF may have a synergistic effect on cancer progression (Oncogene (2007), 26:58-163). See Background for a description of the impact of KRAS mutations on colorectal cancer.

In these methods, the antibodies may be co-administered with another anti-cancer therapy. Suitable anti-cancer therapies for use with the present invention include, but are not limited to, radiation, surgery, chemotherapy, hormonal therapy, immunotherapies, and tyrosine kinase inhibitor therapies, and the like. In some embodiments, the treatment further comprises one of more of: administering a radiation therapy, administering a chemotherapy, or performing surgery.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

In the following example, the inventors describe the expression, purification, and binding and functional characterization of a series of anti-EGFR antibodies produced in wild type, ΔXF and AA N. benthamiana plants. Compared to their CHO expressed counterpart, cetuximab, these plant-produced anti-EGFR antibodies preserved equivalent binding specificity and affinity to EGFR expressing CRC cells. They displayed more uniformed mammalian N-glycosylation of the GnGn and AA glycoforms, thereby improved FcγRIIIa binding and enhanced ADCC activity. Furthermore, antibodies produced in ΔXF and AA plants or with the E430G mutation in the Fc region increased C1q binding and CDC activity. These antibodies may provide new therapeutic options for cancer patients with downstream mutations in the EGFR signaling pathway through improved ADCC and CDC activity.

### Materials and Methods

Expression of Plant Anti-EGFR Antibodies in Wild Type and ΔXF N. benthamiana Leaves

The coding sequences of cetuximab HC and LC were optimized in silico with N. benthamiana-optimized codons as described before (Lai, Engle et al. 2010). The resulting coding sequences synthesized and cloned into the MagnICON plant expression vectors pICH21595 and pICH11599 and then transformed into A. tumefaciens. Wild type or ΔXF N. benthamiana plants were grown and the leaves were co-agroinfiltrated with A. tumefaciens strains containing the HC and LC 5′ modules along with their respective 3′ modules and an integrase construct as described previously for expression of other antibodies (Lai, Engle et al. 2010, He, Lai et al. 2014, Dent, Hurtado et al. 2016).

### Extraction and Purification of Plant Anti-EGFR Antibodies

Agroinfiltrated N. benthamiana leaves were harvested 7 days postinfiltration (dpi). Plant 30 anti-EGFR antibodies were extracted and purified by using a method developed for other plant produced antibodies (Lai, Engle et al. 2010, He, Lai et al. 2014, Lai, He et al. 2014, Dent, Hurtado et al. 2016).

### Gel Electrophoresis and Western Blot

Gel Electrophoresis was carried out by using 10% SDS-PAGE under reducing (5%, v/v, β-mercaptoethanol) or 4-20% gradient SDS-PAGE under non-reducing conditions. Gels were stained with Coomassie blue or used to transfer proteins onto PVDF membranes. HRP-conjugated antibodies against human-kappa LC or gamma HC (Southern Biotech) were used for Western blot analysis as described previously (Lai, He et al. 2014).

### N-Glycan Analysis

LC-ESI-MS was used to determine the N-linked glycosylation profiles of plant produced Anti-EGFR antibodies as described previously (Stadlmann, Pabst et al. 2008). Purified plant produced Anti-EGFR antibodies were first separated by 10% SDS-PAGE using reducing conditions. After gels were stained Coomassie Blue, the HC-containing bands were excised from the gel. Peptide fragments were eluted from the gel with 50% acetonitrile after S-alkylation and tryptic or tryptic/GluC digestion and a reversed-phase column (150×0.32 mm BioBasic-18, Thermo Fisher Scientific) with a 1-80% acetonitrile gradient was used for separation of the peptides. The glycopeptides were analyzed on a quadruple time-of-flight (Q-TOF) Ultima Global mass spectrometer (Waters). Different glycoforms were identified from summed and deconvoluted spectra. The ProGlycAn nomenclature (www.proglycan.com) was used to annotate the glycans.

### Cell Lines

HCT116, HT29 and LS174T cells were cultured in complete McCoy's 5A medium while Caco-2 and A431 cells were cultured in complete Dulbecco's Modified Eagle's Medium (DMEM) according to ATCC instructions.

EGFR binding in CRC cells HT29, HCT116, or LS174T cells (1 million) were collected and washed with 1×PBS before they were stained with either human IgG, CHO expressed anti-EGFR or plant produced anti-EGFR (10 ug/ml). The cells were then washed twice with cold PBS and stained with mouse anti-human antibody conjugated with Alexa488. Finally, the cells were washed twice again and resuspended in 1×PBS. For binding competition with cetuximab, the cells were first incubated with serial dilutions of pAnti-EGFR and washed twice before staining with cetuximab followed by secondary antibody. The fluorescence intensity was measured by flow cytometry (Gallios, Beckman Coulter).

### FcγR Binding Kinetics and Affinity Measurements by Surface Plasmon Resonance

The binding kinetics and affinity of mAbs for Fc receptor CD16 were determined using a Biacore™ X100 instrument (GE Healthcare, Little Chalfont, UK). First, a protein A sensor chip was prepared by immobilizing recombinant protein A (Sigma) in both flow cells of a CM5 sensor chip to 5000 RU using the Amine Coupling Kit (GE Healthcare). MAb samples were diluted in HBS-EP+ buffer and captured onto the Protein A surface to the levels around 330RU. Recombinant human FcγRIIIa ectodomains (R&D) were injected over both flow cells at 25° C. for 135 s at 40 μL/min. The FcγRIIIa-mAb complexes were removed with a 600-s injection of 10 mm glycine-HCl (pH 1.5). Receptors were analyzed at the following concentrations: 0.0625-1 μM. Referenced and blanked sensograms were fitted with BIAcore Evaluation Software using Two State Reaction model.

### ADCC and CDC Assays

ADCC assays were performed using NK cells expanded from human PBMC (Somanchi, Senyukov et al. 2011). Briefly, NK cells were thaw and cultured in complete RPMI medium with human IL-2 the day before the assay. Before adding to the assay plate, target cells were resuspended in complete medium (1×10⁶/ml) with 5 ug/ml Calcein AM (Invitrogen) and incubated for 1 hr at 37 C. In the meantime, NK cells were resuspended at 5×10⁵/ml and transferred to 96-well assay plate at 100ul/well to achieve E:T=5:1. Then the target cells were washed twice with complete medium and incubated with antibodies for 15 minutes before they were added to the assay plate to start the assay. The assay plates were incubated at 37c for 4 hr and centrifuged at 100g for 5 minutes to separate the cells from supernatant. The cell supernatant from each well was transferred to a black and clear bottom 96-well plate and the fluorescence intensity was measure on a Spectramax M5 plate reader (Molecular Devices). The specific lysis rate was calculated as: 100%×(Sample Fluorescence−Spontaneous Fluorescence)/(Maximum Fluorescence−Spontaneous Fluorescence).

CDC assays were performed using A431 cells. Briefly, 0.15 million of A431 cells were plated in each well of 6-well plate. The next day, the media was replaced with 25% normal human serum in DMEM without FBS. Antibodies were added to each well with a final concentration of 20ug/ml. After 48 hrs, the cells were collected and FITC Annexin V staining was performed according to manufacturer's instruction (Biolegend, San Diego, CA). Samples were measured by flow cytometry (Gallios, Beckman Coulter). Relative fold of apoptosis was calculated by the percentage of apoptotic cells from each sample divided by the percentage of apoptotic cells from sample treated with human IgG.

### Results

Expression, Assembly and Purification of Anti-EGFR Antibodies from N. benthamiana

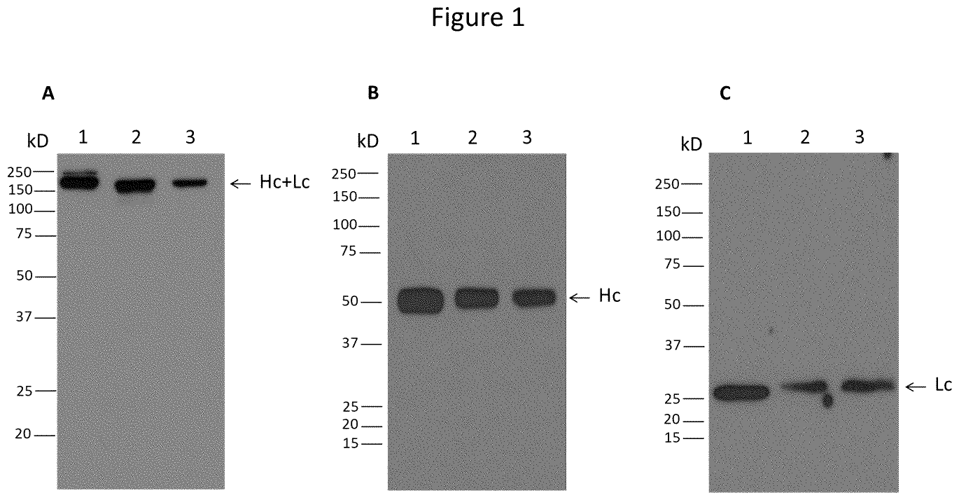

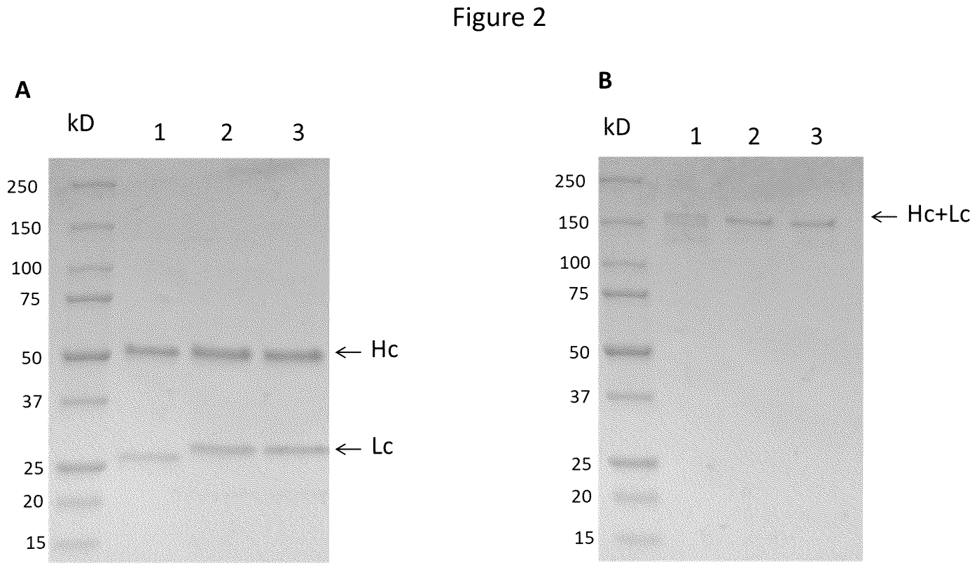

To express anti-EGFR in N. benthamiana, the optimized coding sequences of cetuximab He and Lc were synthesized and cloned into MagnICON plant expression vectors and transformed into Agrobacterium tumefaciens followed by infiltration into N. benthamiana leaves of wild type, ΔXF and AA plants (Strasser, Castilho et al. 2009). FIG. 1 shows the expression profiles of anti-EGFR antibody produced in wild type (wt-Anti-EGFR) and in ΔXF N. benthamiana (ΔXF-Anti-EGFR) analyzed by western blot. Both antibodies were expressed with He and Lc at expected molecular sizes (FIG. 1b and 1c, reducing condition). They were all fully assembled into the heterotetrameric molecules (FIG. 1a, lane 2 and 3) comparable to a positive control (FIG. 1a, lane 1) under non-reducing conditions. Similar results were observed for anti-EGFR expressed in AA plants and mutant antibodies. Plant produced anti-EGFR antibodies were extracted from N. benthamiana leaves and purified by a method previously developed for other plant produced antibodies (Lai, Engle et al. 2010, He, Lai et al. 2014, Lai, He et al. 2014, Dent, Hurtado et al. 2016). The purity of plant produced anti-EGFR antibodies were analyzed by Coomassie blue staining. FIG. 2 demonstrated anti-EGFR antibodies (wt-Anti-EGFR, pAXF-Anti-EGFR and Anti-EGFR E430G) were purified to more than 90% purity from N. benthamiana leaves. Similar purity was achieved for anti-EGFR antibodies expressed in AA plants. Taken together, we have efficiently expressed and purified anti-EGFR antibodies from N. benthamiana plants. Purified wt-Anti-EGFR, pAXF-Anti-EGFR, AA-Anti-EGFR and the E430G mutants were used for functional analyses.

### N-Linked Glycosylation Pattern of Plant Produced Anti-EGFR Antibodies

It is well known that appropriate N-glycosylation is important for human antibody functionality especially Fc domain mediated effector functions. Plant produced antibodies differ from their counterparts produced in mammalian expression system in N-glycosylation diversity and extent (Loos and Steinkellner 2014, Chen 2016). Table 1 summaries the N-glycosylation profiles of plant produced anti-EGFR antibodies and compared to that of CHO cell expressed anti-EGFR (cetuximab). As expected, wt-Anti-EGFR displayed a typical plant N-glycans which contain the plant specific β1,2 xylose and α1,3 fucose. CHO cell expressed anti-EGFR exhibited two major glycoforms: GnGnF6 and AGnF6 both contain the core α1,6 fucosylated structures. The third major glycan of cetuximab mAb is AAF6, which also contain the fucosylated structure. In contrast, one major N-glycan was observed for antibodies produced from glycoengineered plants.

ΔXF-Anti-EGFR was shown to carry the predominant mammalian GnGn structure without the xylose or fucose. AA-Anti-EGFR was shown to carry the P 1,4-galactosylated N-glycan structure as the major glycoform.

Table 1 shows the N-linked glycans of plant produced Anti-EGFR antibodies and CHO cells expressed Anti-EGFR antibody. N-Glycosylation profile was determined by LS-ESI-MS. Numbers represent the presence of the different glycoforms as percentages. Note, ΔXFT is a N. benthamiana N-glycosylation mutant that decorates proteins with mammalian-type GnGn glycans. Glycans were annotated according to the ProGlycAn nomenclature (www.proglycan.com).

| Sample | GnM | GnMXF₃ | GnGnXF₃ | GnGnX | GnGn | GnGnF₆ | AGnF₆ | AAF₆ |
|---|---|---|---|---|---|---|---|---|
| ΔXF-Anti-EGFR | 5.29 | | | | 94.71 | | | |
| WT-Anti-EGFR | | 16.23 | 41.95 | 14.30 | 17.39 | | | |
| CHO-Anti-EGFR | 1.97 | | | | 3.09 | 35.43 | 39.19 | 10.44 |

### Plant Produced Anti-EGFR Antibodies Bind to EGFR Expressing Cancer Cells

To evaluate the binding specificity of plant produced anti-EGFR antibodies, Wt-Anti-EGFR and ΔXF-Anti-EGFR were incubated with human colon cancer cell lines that either express wild-type (WT) Kras (HT29) or mutated Kras (HCT116 and LS174T) oncogene (Hamada, Monnai et al. 2008). The binding specificity was measured by flow cytometry. FIG. 3 shows plant produced anti-EGFR antibodies bind to the EGFR expressing cancer cells equivalently to that of cetuximab, regardless of the Kras mutation status. To investigate if plant-produced Anti-EGFR mAbs recognize the same epitope of EGFR as the reference cetuximab, a competitive flow cytometry experiment was performed. Indeed, wt-Anti-EGFR was able to compete with cetuximab for binding in a dose dependent manner (FIG. 4). These data demonstrate that plant produced anti-EGFR antibodies preserved the binding specificity and affinity to EGFR receptor.

Glycoforms of Plant Produced Anti-EGFR mAbs Exhibits High Affinity Binding to FcγRIIIa (CD16A) and C1q.

N-glycosylation of the Fc region of an antibody directly affects the binding to Fcγ receptors and C1q (Houde, Peng et al. 2010). Surface plasmon resonance (SPR) was used to measure the binding kinetics and affinity of plant produced anti-EGFR antibodies to CD16A and C1q. Compared to the reference cetuximab, ΔXF-Anti-EGFR demonstrated significantly higher association rate but much lower dissociation rate, thereby, resulted in higher affinity binding to CD16A than cetuximab (FIG. 5). Similarly, ΔXF-Anti-EGFR also exhibited higher affinity binding to C1q than that of cetuximab, which may facilitate the activation of the complement cascade. The affinity of AA-Anti-EGFR to CD16A and C1q is similar to that of ΔXF-Anti-EGFR, higher than that of the cetuximab reference.

Plant Produced Anti-EGFR Antibodies Elicit Enhanced ADCC and CDC Activity Against CRC Cells with Both Wt and Mutant Kras Genes

ADCC assay was carried out to examine whether the plant produced anti-EGFR antibodies could enhance cancer cell killing through the enhanced binding affinity to FcγRIII on NK cells compared to that of cetuximab. Plant-produced ΔXF-Anti-EGFR and ΔXF-Anti-EGFR E430G (with Fc amino acid mutation E430G) exhibited superior cancer cell lysis rates than cetuximab at the same conditions using either Caco-2 cells (wt Kras, FIG. 6A) or HCT116 cells (Kras mutant, FIG. 6B). In addition to ΔXF-Anti-EGFR, plant-produced Anti-EGFR mAbs with glycosylation form of AA and with combination of Fc amino acid mutation E430G (wt-E430G, AA-E430G) also have superior ADCC activity over cetuximab (data not shown). Similar results were obtained with HT29 cells (Wt Kras, Braf mutant, data not shown). As CRC patients with downstream mutations such as Kras in the EGFR signaling pathway are not responsive to CHO cell-produced cetuximab, the enhanced killing of cancer cells by plant-produced anti-EGFR antibodies via NK cell-mediated ADCC may provide an alternative mechanism of treatment for these patients.

To evaluate whether our plant produced anti-EGFR antibodies could also enhance the CDC activity, EGFR-expressing A431 cancer cells were incubated with plant-produced anti-EGFR mAbs, cetuximab or human IgG isotype control and fresh normal human serum as a source of complement. The CDC activity of various mAbs was measured by flow cytometry for Annexin V staining positive cells. Except wt-anti-EGFR, all plant-produced anti-EGFR antibodies including ΔXF-Anti-EGFR and AA-Anti-EGFR exhibited higher CDC activity of A431 cancer cells over that of cetuximab (FIG. 7). In addition, Fc E430G mutation further increased the CDC activity of plant-produced anti-EGFR mAbs (ΔXF-Anti-EGFR E430G and AA-Anti-EGFR E430G) and even including the one with WT plant glycans (WT-Anti-EGFR E430G) (FIG. 7). Taken together, our plant-produced anti-EGFR antibodies not only preserved the binding affinity and specificity of cetuximab, but also have improved ADCC and CDC activities against cancer cells irrespective of KRAS mutation status.

### Discussion

Since the first cancer antibody drug Rituximab was approved by FDA in 1997, many monoclonal antibodies have been developed and become an important category of therapeutics against various types of cancers (Chiavenna, Jaworski et al. 2017). Cetuximab as one of the most used monoclonal antibodies in clinical practice (Chiavenna, Jaworski et al. 2017), has been successful for treatment of metastatic CRC (Sobani, Sawant et al. 2016). Despite that, increasing evidence suggested that CRC patients with any mutations in the downstream of the EGFR signaling pathways, including Kras, Braf and PI3K, may have poor response to anti-EGFR therapy (Sobani, Sawant et al. 2016). Analysis of gene mutations in metastatic CRC tumors indicated that about 30-40% of tumors have Kras mutation, 15% have PI3K mutation and 3-5% have Braf mutation (De Roock, Claes et al. 2010, Pentheroudakis, Kotoula et al. 2013). These mutations significantly limited the benefit of anti-EGFR antibodies for metastatic CRC treatment. It also highlights the urgency of developing new antibody therapeutics to benefit CRC patients with Kras, Braf or PI3K mutations.

Here we described the expression, purification and functional characterization of a series of anti-EGFR antibodies produced from wild type and glycoengineered N. benthamiana Tabaco plants. All our plant produced anti-EGFR antibodies preserved the EGFR binding affinity and specificity in both wild type and mutant Kras cancer cell lines equivalent to their mammalian expressed counterpart cetuximab. More importantly, they all induced higher ADCC activity by NK cells for killing wild type, Kras mutant or Braf mutant CRC cells than cetuximab. Furthermore, the anti-EGFR antibodies produced in the two glycoengineered plants or wild type plants with the E430G mutation displayed more potent CDC activity than cetuximab in a cancer cell line with Braf mutation. These antibodies may benefit all patients with metastatic CRC regardless of the mutation status in the downstream of EGFR signaling pathways. Improving effector functions has become an important approach to optimize cancer antibody therapeutics (Natsume, Niwa et al. 2009, Weiner 2015, Wirt, Rosskopf et al. 2017). However, it could be difficult to enhance both ADCC and CDC activity simultaneously for one single antibody. Our data demonstrated that by using glycoengineered N. benthamiana plants, this can be achieved in a relatively straightforward process, even the mutations in the Fc region to promote C1q binding may not be necessary.

It is well accepted that ADCC activity plays a crucial role in antibody immunotherapy (Weiner 2018). Patients with the FcγRIIIa 158V genotype tend to have more potent ADCC activity than the FcγRIIIa 158F genotype probably due to the tighter binding to IgGI (Koene, Kleijer et al. 1997). Nevertheless, the enhancement of ADCC through Fc defucosylation is independent of the FcγRIIIa phenotypes (Niwa, Hatanaka et al. 2004). Our anti-EGFR antibodies produced in glycoengineered plants are not only defucosylated but consist of only one predominant N-glycan form, either GnGn or AA. Both sugar moieties may contribute to the enhancement of ADCC activity (Umana, Jean-Mairet et al. 1999, Zeitlin, Pettitt et al. 2011, Thomann, Reckermann et al. 2016).

Early in vitro and in vivo studies indicate that complement membrane attack complex (MAC) may induce apoptosis and contribute to apoptotic cell death under pathological conditions (Fishelson, Attali et al. 2001). Several studies have provided evidence that the activation of the complement system can induce apoptosis and contribute to apoptotic cell death (Nauta, Daha et al. 2002, Flierl, Rittirsch et al. 2008, Hong, Sze et al. 2009, Kaur, Sultan et al. 2016). Our CDC assay measured the outcome of the treatment of anti-EGFR antibodies and normal human serum for A431 cells over 48 hr time period. The results show that all of our plant produced antibodies except that from wild type plants induced more apoptosis compared to cetuximab in A431 cells treated with 25% normal human serum. Inducing apoptosis is one of the mechanisms of action for cetuximab in anti-cancer therapy (Vincenzi, Schiavon et al. 2008). Our plant produced antibodies do not change the binding affinity for EGFR. But they can enhance complement activation either through formation of the hexamers after binding to EGFR for E430G mutants (De Roock, Claes et al. 2010) or through the terminal galactoses increasing binding to C1q for antibody generated in AA plants (Raju 2008).

Compared to expression platforms based on mammalian cells, such as CHO cells, plants have a much smaller glycome and have exceptional tolerance for glycan modifications (Kallolimath and Steinkellner 2015, Chen 2016) which make them very valuable for producing glycoproteins with defined and homogenous N-glycans. In addition, plant expression systems could provide high speed and high yield antibody production with scalability (Chen 2016). Our anti-EGFR antibodies produced in glycoengineered N. benthamiana plants with improved ADCC and CDC activities, demonstrate that plant glycoengineering could be a feasible approach towards generation of optimized antibodies for cancer immunotherapy.

### REFERENCES

Buswell, S., F. Medina-Bolivar, Q. Chen, K. Van Cott and C. Zhang (2005). “Expression of porcine prorelaxin in transgenic tobacco.” Ann N Y Acad Sci 1041: 77-81.
Chen, Q. (2016). “Glycoengineering of plants yields glycoproteins with polysialylation and other defined N-glycoforms.” Proc Natl Acad Sci USA 113(34): 9404-9406.
Chen, Q. a. D., K R (2016). “The potential of plants as a system for the development and production of human biologics [version 1; referees: 3 approved]..” F1000Research 5.
Chiavenna, S. M., J. P. Jaworski and A. Vendrell (2017). “State of the art in anti-cancer mAbs.” Journal of Biomedical Science 24.
de Jong, R. N., F. J. Beurskens, S. Verploegen, K. Strumane, M. D. van Kampen, M. Voorhorst, W. Horstman, P. J. Engelberts, S. C. Oostindie, G. B. Wang, A. J. R. Heck, J. Schuurman and P. W. H. I. Parren (2016). “A Novel Platform for the Potentiation of Therapeutic Antibodies Based on Antigen-Dependent Formation of IgG Hexamers at the Cell Surface.” Plos Biology 14(1).
De Roock, W., B. Claes, D. Bernasconi, J. De Schutter, B. Biesmans, G. Fountzilas, K. T. Kalogeras, V. Kotoula, D. Papamichael, P. Laurent-Puig, F. Penault-Llorca, P. Rougier, B. Vincenzi, D. Santini, G. Tonini, F. Cappuzzo, M. Frattini, F. Molinari, P. Saletti, S. De Dosso, M. Martini, A. Bardelli, S. Siena, A. Sartore-Bianchi, J. Tabernero, T. Macarulla, F. Di Fiore, A. O. Gangloff, F. Ciardiello, P. Pfeiffer, C. Qvortrup, T. P. Hansen, E. Van Cutsem, H. Piessevaux, D. Lambrechts, M. Delorenzi and S. Tejpar (2010). “Effects of KRAS, BRAF, NRAS, and PIK3CA mutations on the efficacy of cetuximab plus chemotherapy in chemotherapy-refractory metastatic colorectal cancer: a retrospective consortium analysis.” Lancet Oncology 11(8): 753-762.
Dent, M., J. Hurtado, A. M. Paul, H. Sun, H. Lai, M. Yang, A. Esqueda, F. Bai, H. Steinkellner and Q. Chen (2016). “Plant-produced anti-dengue virus monoclonal antibodies exhibit reduced antibody-dependent enhancement of infection activity.” J Gen Virol 97(12): 3280-3290.
Diebolder, C. A., F. J. Beurskens, R. N. de Jong, R. I. Koning, K. Strumane, M. A. Lindorfer, M. Voorhorst, D. Ugurlar, S. Rosati, A. J. R. Heck, J. G. J. van de Winkel, I. A. Wilson, A. J. Koster, R. P. Taylor, E. O. Saphire, D. R. Burton, J. Schuurman, P. Gros and P. W. H. I. Parren (2014). “Complement Is Activated by IgG Hexamers Assembled at the Cell Surface.” Science 343(6176): 1260-1263.
Douillard, J. Y., K. S. Oliner, S. Siena, J. Tabernero, R. Burkes, M. Barugel, Y. Humblet, G. Bodoky, D. Cunningham, J. Jassem, F. Rivera, I. Kocakova, P. Ruff, M. Blasinska-Morawiec, M. Smakal, J. L. Canon, M. Rother, R. Williams, A. Rong, J. Wiezorek, R. Sidhu and S. D. Patterson (2013). “Panitumumab-FOLFOX4 treatment and RAS mutations in colorectal cancer.” N Engl J Med 369(11): 1023-1034.
Fishelson, Z., G. Attali and D. Mevorach (2001). “Complement and apoptosis.” Molecular Immunology 38(2-3): 207-219.
Flierl, M. A., D. Rittirsch, A. J. Chen, B. A. Nadeau, D. E. Day, J. V. Sarma, M. S. Huber-Lang and P. A. Ward (2008). “The Complement Anaphylatoxin C5a Induces Apoptosis in Adrenomedullary Cells during Experimental Sepsis.” Plos One 3(7).
Hamada, K., M. Monnai, K. Kawai, C. Nishime, C. Kito, N. Miyazaki, Y. Ohnishi, M. Nakamura and H. Suemizu (2008). “Liver metastasis models of colon cancer for evaluation of drug efficacy using NOD/Shi-scid IL2Rynull (NOG) mice.” mt J Oncol 32(1): 153-159.
He, J., H. Lai, M. Engle, S. Gorlatov, C. Gruber, H. Steinkellner, M. S. Diamond and Q. Chen (2014). “Generation and analysis of novel plant-derived antibody-based therapeutic molecules against West Nile virus.” PLoS One 9(3): e93541.
Hong, Q. Y., C. I. Sze, S. R. Lin, M. H. Lee, R. Y. He, L. Schultz, J. Y. Chang, S. J. Chen, R. J. Boackle, L. J. Hsu and N. S. Chang (2009). “Complement C1q Activates Tumor Suppressor WWOX to Induce Apoptosis in Prostate Cancer Cells.” Plos One 4(6).
Houde, D., Y. Peng, S. A. Berkowitz and J. R. Engen (2010). “Post-translational modifications differentially affect IgG1 conformation and receptor binding.” Mol Cell Proteomics 9(8): 1716-1728.
Kallolimath, S. and H. Steinkellner (2015). “Glycosylation of plant produced human antibodies.” Hum Antibodies 23(3-4): 45-48.
Karapetis, C. S., S. Khambata-Ford, D. J. Jonker, C. J. O'Callaghan, D. Tu, N. C. Tebbutt, R. J. Simes, H. Chalchal, J. D. Shapiro, S. Robitaille, T. J. Price, L. Shepherd, H. J. Au, C. Langer, M. J. Moore and J. R. Zalcberg (2008). “K-ras mutations and benefit from cetuximab in advanced colorectal cancer.” N Engl J Med 359(17): 1757-1765.
Kaur, A., S. H. A. Sultan, V. Murugaiah, A. A. Pathan, F. S. Alhamlan, E. Karteris and U. Kishore (2016). “Human C1q Induces Apoptosis in an Ovarian Cancer Cell Line via Tumor Necrosis Factor Pathway.” Frontiers in Immunology 7.
Koene, H. R., M. Kleijer, J. Algra, D. Roos, A. E. von dem Borne and M. de Haas (1997). “Fc gammaRIIIa-158V/F polymorphism influences the binding of IgG by natural killer cell Fc gammaRIIIa, independently of the Fc gammaRIIIa-48L/R/H phenotype.” Blood 90(3): 1109-1114.
Lai, H., M. Engle, A. Fuchs, T. Keller, S. Johnson, S. Gorlatov, M. S. Diamond and Q. Chen (2010). “Monoclonal antibody produced in plants efficiently treats West Nile virus infection in mice.” Proc Natl Acad Sci USA 107(6): 2419-2424.
Lai, H., J. He, J. Hurtado, J. Stahnke, A. Fuchs, E. Mehlhop, S. Gorlatov, A. Loos, M. S. Diamond and Q. Chen (2014). “Structural and functional characterization of an anti-West Nile virus monoclonal antibody and its single-chain variant produced in glycoengineered plants.” Plant Biotechnol J 12(8): 1098-1107.
Li, T., D. J. DiLillo, S. Bournazos, J. P. Giddens, J. V. Ravetch and L. X. Wang (2017). “Modulating IgG effector function by Fc glycan engineering.” Proc Natl Acad Sci USA 114(13): 3485-3490.
Lievre, A., J. Bachet, V. Boige, B. Landi, J. Emile, J. Cote, G. Tomasic, P. Rougier, F. Penault-Llorca and P. Laurent-Puig (2006). “KRAS mutation status is predictive of response to cetuximab therapy in colorectal cancer.” Annals of Oncology 17: 42-42.
Loos, A. and H. Steinkellner (2014). “Plant glyco-biotechnology on the way to synthetic biology.” Front Plant Sci 5: 523.
Marusic, C., C. Pioli, S. Stelter, F. Novelli, C. Lonoce, E. Morrocchi, E. Benvenuto, A. M. Salzano, A. Scaloni and M. Donini (2018). “N-glycan engineering of a plant-produced anti-CD20-hIL-2 immunocytokine significantly enhances its effector functions.” Biotechnol Bioeng 115(3): 565-576.
Natsume, A., R. Niwa and M. Satoh (2009). “Improving effector functions of antibodies for cancer treatment: Enhancing ADCC and CDC.” Drug Des Devel Ther 3: 7-16.
Nauta, A. J., M. R. Daha, O. Tijsma, B. Van de Water, F. Tedesco and A. Roos (2002). “The membrane attack complex of complement induces caspase activation and apoptosis.” European Journal of Immunology 32(3): 783-792.
NCI “National Cancer Institute. SEER cancer statistics factsheets: colon and rectum cancer. 2016 ” https://seer.cancer.gov/statfacts/html/colorect.html. Niwa, R., S. Hatanaka, E. Shoji-Hosaka, M. Sakurada, Y. Kobayashi, A. Uehara, H.
Yokoi, K. Nakamura and K. Shitara (2004). “Enhancement of the antibody-dependent cellular cytotoxicity of low-fucose IgGI Is independent of FcgammaRIIIa functional polymorphism.” Clin Cancer Res 10(18 Pt 1): 6248-6255.
Niwa, R., E. Shoji-Hosaka, M. Sakurada, T. Shinkawa, K. Uchida, K. Nakamura, K. Matsushima, R. Ueda, N. Hanai and K. Shitara (2004). “Defucosylated chimeric anti-CC chemokine receptor 4 IgGI with enhanced antibody-dependent cellular cytotoxicity shows potent therapeutic activity to T-cell leukemia and lymphoma.” Cancer Research 64(6): 2127-2133.
Pentheroudakis, G., V. Kotoula, W. De Roock, G. Kouvatseas, P. Papakostas, T. Makatsoris, D. Papamichael, I. Xanthakis, J. Sgouros, D. Televantou, G. Kafiri, A. C. Tsamandas, E. Razis, E. Galani, D. Bafaloukos, I. Efstratiou, I. Bompolaki, D. Pectasides, N. Pavlidis, S. Tejpar and G. Fountzilas (2013). “Biomarkers of benefit from cetuximab-based therapy in metastatic colorectal cancer: interaction of EGFR ligand expression with RAS/RAF, PIK3CA genotypes.” Bmc Cancer 13.
Prior, I. A., P. D. Lewis and C. Mattos (2012). “A comprehensive survey of Ras mutations in cancer.” Cancer Res 72(10): 2457-2467.
Q, C. and D. KR. (2016). “The potential of plants as a system for the development and production of human biologics [version 1; referees: 3 approved]. F1000Research 2016, 5(F1000 Faculty Rev):912 (doi: 10.12688/fl000research.8010.1).” F1000Research: 912.
Qiu, X., G. Wong, J. Audet, A. Bello, L. Fernando, J. B. Alimonti, H. Fausther-Bovendo, H. Wei, J. Aviles, E. Hiatt, A. Johnson, J. Morton, K. Swope, O. Bohorov, N. Bohorova, C. Goodman, D. Kim, M. H. Pauly, J. Velasco, J. Pettitt, G. G. Olinger, K. Whaley, B. Xu, J. E. Strong, L. Zeitlin and G. P. Kobinger (2014). “Reversion of advanced Ebola virus disease in nonhuman primates with ZMapp.” Nature 514(7520): 47-53.
Raju, T. S. (2008). “Terminal sugars of Fc glycans influence antibody effector functions of IgGs.” Current Opinion in Immunology 20(4): 471-478.
Scheffzek, K., M. R. Ahmadian, W. Kabsch, L. Wiesmuller, A. Lautwein, F. Schmitz and A. Wittinghofer (1997). “The Ras-RasGAP complex: structural basis for GTPase activation and its loss in oncogenic Ras mutants.” Science 277(5324): 333-338.
Shields, R. L., J. Lai, R. Keck, L. Y. O'Connell, K. Hong, Y. G. Meng, S. H. A. Weikert and L. G. Presta (2002). “Lack of fucose on human IgGI N-linked oligosaccharide improves binding to human Fc gamma RIII and antibody-dependent cellular toxicity.” Journal of Biological Chemistry 277(30): 26733-26740.
Shinkawa, T., K. Nakamura, N. Yamane, E. Shoji-Hosaka, Y. Kanda, M. Sakurada, K. Uchida, H. Anazawa, M. Satoh, M. Yamasaki, N. Hanai and K. Shitara (2003). “The absence of fucose but not the presence of galactose or bisecting N-acetylglucosamine of human IgGI complex-type oligosaccharides shows the critical role of enhancing antibody-dependent cellular cytotoxicity.” Journal of Biological Chemistry 278(5): 3466-3473.
Sobani, Z. A., A. Sawant, M. Jafri, A. K. Correa and I. H. Sahin (2016). “Oncogenic fingerprint of epidermal growth factor receptor pathway and emerging epidermal growth factor receptor blockade resistance in colorectal cancer.” World J Clin Oncol 7(5): 340-351.
Somanchi, S. S., V. V. Senyukov, C. J. Denman and D. A. Lee (2011). “Expansion, purification, and functional assessment of human peripheral blood NK cells.” J Vis Exp(48).
Stadlmann, J., M. Pabst, D. Kolarich, R. Kunert and F. Altmann (2008). “Analysis of immunoglobulin glycosylation by LC-ESI-MS of glycopeptides and oligosaccharides.” Proteomics 8(14): 2858-2871.
Strasser, R., A. Castilho, J. Stadlmann, R. Kunert, H. Quendler, P. Gattinger, J. Jez, T. Rademacher, F. Altmann, L. Mach and H. Steinkellner (2009). “Improved virus neutralization by plant-produced anti-HIV antibodies with a homogeneous beta1,4-galactosylated N-glycan profile.” J Biol Chem 284(31): 20479-20485.
Therkildsen, C., T. K. Bergmann, T. Henrichsen-Schnack, S. Ladelund and M. Nilbert (2014). “The predictive value of KRAS, NRAS, BRAF, PIK3CA and PTEN for anti-EGFR treatment in metastatic colorectal cancer: A systematic review and meta-analysis.” Acta Oncol 53(7): 852-864.
Thomann, M., K. Reckermann, D. Reusch, J. Prasser and M. L. Tejada (2016). “Fc-galactosylation modulates antibody-dependent cellular cytotoxicity of therapeutic antibodies.” Molecular Immunology 73: 69-75.
Tuse, D., T. Tu and K. A. McDonald (2014). “Manufacturing economics of plant-made biologics: case studies in therapeutic and industrial enzymes.” Biomed Res Int 2014:256135.
Umana, P., J. Jean-Mairet, R. Moudry, H. Amstutz and J. E. Bailey (1999). “Engineered glycoforms of an antineuroblastoma IgGI with optimized antibody-dependent cellular cytotoxic activity.” Nat Biotechnol 17(2): 176-180.
Vincenzi, B., G. Schiavon, M. Silletta, D. Santini and G. Tonini (2008). “The biological properties of cetuximab.” Critical Reviews in Oncology Hematology 68(2): 93-106.
Weiner, D. Z. D. A. A. O. C. L. M. (2018). “Enhancing antibody-dependent cell-mediated cytotoxicity: a strategy for improving antibody-based immunotherapy.” Antibody Therapeutics 1(1): 7-12.
Weiner, G. J. (2015). “Building better monoclonal antibody-based therapeutics.” Nat Rev Cancer 15(6): 361-370.
Wirt, T., S. Rosskopf, T. Rosner, K. M. Eichholz, A. Kahrs, S. Lutz, A. Kretschmer, T. Valerius, K. Klausz, A. Otte, M. Gramatzki, M. Peipp and C. Kellner (2017). “An Fc Double-Engineered CD20 Antibody with Enhanced Ability to Trigger Complement-Dependent Cytotoxicity and Antibody-Dependent Cell-Mediated Cytotoxicity.” Transfus Med Hemother 44(5): 292-300.
Zeitlin, L., J. Pettitt, C. Scully, N. Bohorova, D. Kim, M. Pauly, A. Hiatt, L. Ngo, H. Steinkellner, K. J. Whaley and G. G. Olinger (2011). “Enhanced potency of a fucose-free monoclonal antibody being developed as an Ebola virus immunoprotectant.” Proceedings of the National Academy of Sciences of the United States of America 108(51): 20690-20694.

### Embodiments of the Invention

1. An anti-EGFR antibody comprising: a heavy chain comprising a polypeptide with at least 95% sequence identity to SEQ ID NO:2 and a light chain comprising a polypeptide with at least 95% sequence identity to SEQ ID NO:4, wherein the antibody:
a) has a glycosylation pattern selected from the group consisting of: GnGn, AA, and GnGnXF;
b) comprises an E430G mutation in the fragment crystallizable (Fc) region; or
c) both (a) and (b); 20 wherein SEQ ID NO:2 is optionally encoded by SEQ ID NO:1 and SEQ ID NO:4 is optionally encoded by SEQ ID NO: 3.

2. The antibody of embodiment 1, wherein the heavy chain and the light chain each further comprise a plant signal peptide.

3. The antibody of embodiment 2, wherein the plant signal peptide comprises SEQ ID NO:6.

4. The antibody of any one of the proceeding embodiments, wherein the heavy chain comprises SEQ ID NO:8 or SEQ ID NO:10 and the light chain comprises SEQ ID NO:12; wherein SEQ ID NO:8 is optionally encoded by SEQ ID NO:7, SEQ ID NO: 10 is optionally encoded by SEQ ID NO:9, and SEQ ID NO:12 is optionally encoded by SEQ ID NO:11.

5. A plant expression vector encoding an anti-EGFR antibody comprising: a heavy chain comprising SEQ ID NO:2 and a light chain comprising SEQ ID NO:4.

6. The plant expression vector of embodiment 5, wherein the heavy chain and the light chain each further comprise a plant signal peptide.

7. The plant expression vector of embodiments 5 or 6, wherein the plant signal peptide comprises SEQ ID NO:6.

8. The plant expression vector of any one of embodiments 5-7, wherein the heavy chain comprises SEQ ID NO:8 or SEQ ID NO:10 and the light chain comprises SEQ ID NO:12.

9. The plant expression vector of any one of embodiments 5-8, wherein the expression vector backbone is MagnICON®.

10. A plant transformed with the plant expression vector of any one of embodiments 5-9.

11. The plant of embodiment 10, wherein the plant is Nicotiana benthamiana.

12. The plant of embodiment 11, wherein the plant is a ΔXF plant or an AA plant.

13. The plant of embodiment 12, wherein the plant expresses 1-2 dominant glycoforms of the antibody encoded by the plant expression vector.

14. An anti-EGFR antibody produced by the plant of any one of embodiments 10-13.

15. The antibody of embodiment 14, wherein the antibody has a glycosylation patterns selected from the group consisting of: GnGn, AA, and GnGnXF.

16. The antibody of any one of embodiments 1-4 or 14-15, wherein the antibody binds to cancer cells that express a wild-type KRas protein or a mutated KRas protein.

17. The antibody of any one of embodiments 1-4 or 14-16, wherein the antibody binds to the same epitope of EGFR as cetuximab.

18. The antibody of any one of embodiments 1-4 or 14-17, wherein the antibody has a higher affinity to cluster of differentiation 16A (CD16A) than cetuximab.

19. The antibody of any one of embodiments 1-4 or 14-18, wherein the antibody has a higher affinity to complement component 1q (C1q) than cetuximab.

20. The antibody of any one of embodiments 1-4 or 14-19, wherein the antibody has a higher antibody dependent cellular cytotoxicity (ADCC) activity than cetuximab.

21. The antibody of any one of embodiments 1-4 or 14-20, wherein the antibody has higher complement dependent cytotoxicity (CDC) activity than cetuximab.

22. The antibody of any one of embodiments 1-4 or 14-21, wherein the heavy chain comprises SEQ ID NO:8 or SEQ ID NO:10 and the light chain comprises SEQ ID NO:12, and wherein the antibody has a glycosylation patterns selected from the group consisting of: GnGn, AA, and GnGnXF.

22. A method of treating cancer in a subject by administering the antibody of embodiments 1-4 or 14-21.

23. The method of embodiment 22, wherein the cancer is selected from colorectal cancer, non-small cell lung cancer, and head/neck cancer.

24. The method of embodiments 22 or 23, wherein the cancer comprises a mutation in KRAS or BRAF.

25. The method of any one of embodiments 22-24, wherein the treatment further comprises one of more of: administering a radiation therapy, administering a chemotherapy, or performing surgery

1. An anti-EGFR antibody comprising: a heavy chain comprising a polypeptide with at least 95% sequence identity to SEQ ID NO:2 and a light chain comprising a polypeptide with at least 95% sequence identity to SEQ ID NO:4, wherein the antibody:
a) has a glycosylation pattern selected from the group consisting of: GnGn, AA, and GnGnXF;
b) comprises an E430G mutation in the fragment crystallizable (Fc) region; or
c) both (a) and (b);
wherein SEQ ID NO:2 is optionally encoded by SEQ ID NO:1 and SEQ ID NO:4 is optionally encoded by SEQ ID NO: 3.

2. The antibody of claim 1, wherein the heavy chain and the light chain each further comprise a plant signal peptide.

3. (canceled)

4. The antibody of claim 1, wherein the heavy chain comprises SEQ ID NO:8 or SEQ ID NO:10 and the light chain comprises SEQ ID NO:12; wherein SEQ ID NO:8 is optionally encoded by SEQ ID NO:7, SEQ ID NO:10 is optionally encoded by SEQ ID NO:9, and SEQ ID NO:12 is optionally encoded by SEQ ID NO:11.

5. A plant expression vector encoding an anti-EGFR antibody comprising: a heavy chain comprising SEQ ID NO:2 and a light chain comprising SEQ ID NO:4.

6. The plant expression vector of claim 5, wherein the heavy chain and the light chain each further comprise a plant signal peptide.

7. (canceled)

8. The plant expression vector of claim 5, wherein the heavy chain comprises SEQ ID NO:8 or SEQ ID NO:10 and the light chain comprises SEQ ID NO:12.

9. The plant expression vector of claim 5, wherein the expression vector backbone comprises a plant virus-based expression system.

10. A plant transformed with the plant expression vector of claim 5.

11. The plant of claim 10, wherein the plant is Nicotiana benthamiana.

12. The plant of claim 11, wherein the plant is a ΔXF plant or an AA plant.

13. (canceled)

14. An anti-EGFR antibody produced by the plant of claim 10.

15. The antibody of claim 14, wherein the antibody has a glycosylation pattern selected from the group consisting of: GnGn, AA, and GnGnXF.

16. (canceled)

17. The antibody of claim 1, wherein the antibody binds to the same epitope of EGFR as cetuximab.

18. The antibody of claim 1, wherein the antibody has a higher affinity to cluster of differentiation 16A (CD16A) than cetuximab.

19. (canceled)

20. The antibody of claim 1, wherein the antibody has a higher antibody dependent cellular cytotoxicity (ADCC) activity than cetuximab.

21. The antibody of claim 1, wherein the antibody has higher complement dependent cytotoxicity (CDC) activity than cetuximab.

22. (canceled)

22. A method of treating cancer in a subject by administering the antibody of claim 1.

23. The method of claim 22, wherein the cancer is selected from colorectal cancer, non-small cell lung cancer, and head/neck cancer.

24. The method of claim 22, wherein the cancer comprises a mutation in KRAS or BRAF.

25. (canceled)

26. The antibody of claim 1, wherein the heavy chain comprises SEQ ID NO:8 or SEQ ID NO:10 and the light chain comprises SEQ ID NO:12, and wherein the antibody has a glycosylation pattern selected from the group consisting of: GnGn, AA, and GnGnXF.


SEQUENCE LISTING (12 sequences)

SEQ ID NO: 1 - DNA, 357 bp, Artificial Sequence
  Synthetic- Heavy chain variable domain (VH)
caagtgcaattgaaacagtccggcccaggattggtccagccatcgcaaagtctctctattacatgtaccgttagcggtttttctcttactaactatggggttcattgggtcagacagtcacctgggaaaggattagagtggcttggcgttatctggtctggtggaaatactgactacaatactccctttacatcaaggctatccattaacaaggataactctaagagccaagtgttcttcaagatgaatagtctgcagagtaatgatacggctatatactattgcgcacgtgccttgacttactatgattatgaatttgcttactggggtcaaggaacacttgtaaccgtatcagca

SEQ ID NO: 2 - protein, 119 aa, Artificial Sequence
  Synthetic- Heavy chain variable domain (VH)
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 3 - DNA, 321 bp, Artificial Sequence
  Synthetic- Light chain variable domain (VL)
gatattctcctaacgcaaagtcccgtaattctgagtgtctcccctggggaaagggtgtcgtttagctgtcgagcttctcaatccattggtacaaacatacattggtaccagcagagaactaatggttctcctcgtttattgatcaaatatgcatctgagtcaatttctggcataccatcaagattctctggaagcggttcaggaactgactttaccctttcaatcaatagtgttgaatccgaggatattgccgattactattgccagcaaaacaacaattggccaacaactttcggagctggcacaaagttggaacttaaa

SEQ ID NO: 4 - protein, 107 aa, Artificial Sequence
  Synthetic- Light chain variable domain (VL)
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 5 - DNA, 57 bp, Artificial Sequence
  Synthetic-Plant signal peptide
atgggatggtcttgtatcatccttttcttggttgcaacagctactggtgttcattct

SEQ ID NO: 6 - protein, 19 aa, Artificial Sequence
  Synthetic- Plant signal peptide
MGWSCIILFLVATATGVHS

SEQ ID NO: 7 - DNA, 1452 bp, Artificial Sequence
  Synthetic- Heavy chain, wild-type
gaattcttttggtctcactagacaatgggatggtcttgtatcatccttttcttggttgcaacagctactggtgttcattctcaagtgcaattgaaacagtccggcccaggattggtccagccatcgcaaagtctctctattacatgtaccgttagcggtttttctcttactaactatggggttcattgggtcagacagtcacctgggaaaggattagagtggcttggcgttatctggtctggtggaaatactgactacaatactccctttacatcaaggctatccattaacaaggataactctaagagccaagtgttcttcaagatgaatagtctgcagagtaatgatacggctatatactattgcgcacgtgccttgacttactatgattatgaatttgcttactggggtcaaggaacacttgtaaccgtatcagcagctagcaccaagggaccttctgtttttccacttgctccttcttctaagtctacttctggtggaactgctgctttgggttgtttggtgaaagattactttcctgagccagtgaccgtttcttggaactcaggtgctcttacatctggtgttcatactttcccagctgttcttcaatcttcaggactttactcactttcttctgttgttaccgttccttcttcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtgtccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatgaggctcttcacaatcactacacacagaagagtctctccttgtctccgggtaaatgaagctagagaccaaaaggatcc

SEQ ID NO: 8 - protein, 468 aa, Artificial Sequence
  Synthetic- Heavy chain, wild-type
MGWSCIILFLVATATGVHSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1452 bp, Artificial Sequence
  Synthetic- Heavy chain, E430G mutant
  misc_feature (1375)..(1377): GAG to GGT mutation
gaattcttttggtctcactagacaatgggatggtcttgtatcatccttttcttggttgcaacagctactggtgttcattctcaagtgcaattgaaacagtccggcccaggattggtccagccatcgcaaagtctctctattacatgtaccgttagcggtttttctcttactaactatggggttcattgggtcagacagtcacctgggaaaggattagagtggcttggcgttatctggtctggtggaaatactgactacaatactccctttacatcaaggctatccattaacaaggataactctaagagccaagtgttcttcaagatgaatagtctgcagagtaatgatacggctatatactattgcgcacgtgccttgacttactatgattatgaatttgcttactggggtcaaggaacacttgtaaccgtatcagcagctagcaccaagggaccttctgtttttccacttgctccttcttctaagtctacttctggtggaactgctgctttgggttgtttggtgaaagattactttcctgagccagtgaccgtttcttggaactcaggtgctcttacatctggtgttcatactttcccagctgttcttcaatcttcaggactttactcactttcttctgttgttaccgttccttcttcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtgtccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatggtgctcttcacaatcactacacacagaagagtctctccttgtctccgggtaaatgaagctagagaccaaaaggatcc

SEQ ID NO: 10 - protein, 468 aa, Artificial Sequence
  Synthetic- Heavy chain, E430G mutant
  MISC_FEATURE (451)..(451): Mutation from E to G
MGWSCIILFLVATATGVHSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 11 - DNA, 732 bp, Artificial Sequence
  Synthetic- Light chain
gaattctctagaacaatgggatggtcttgtatcatccttttcttggttgcaacagctactggtgttcattctgatattctcctaacgcaaagtcccgtaattctgagtgtctcccctggggaaagggtgtcgtttagctgtcgagcttctcaatccattggtacaaacatacattggtaccagcagagaactaatggttctcctcgtttattgatcaaatatgcatctgagtcaatttctggcataccatcaagattctctggaagcggttcaggaactgactttaccctttcaatcaatagtgttgaatccgaggatattgccgattactattgccagcaaaacaacaattggccaacaactttcggagctggcacaaagttggaacttaaagctagcagaactgttgctgcaccatctgttttcatcttccctccatctgatgagcagttgaaatctggaactgcttctgttgtgtgccttcttaataacttctatcctagagaggctaaagttcagtggaaggtggataacgcacttcaatctggtaactctcaagagtctgttacagagcaagattctaaggactcaacttactctctttcatctacacttactttgtcaaaagcagattacgagaaacacaaagtttacgcatgcgaagttactcatcaaggactttcttcaccagttacaaagtctttcaatagaggagagtgttaatgagagctc

SEQ ID NO: 12 - protein, 235 aa, Artificial Sequence
  Synthetic- Light chain
  MISC_FEATURE (127)..(128): AS Linker
MGWSCIILFLVATATGVHSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKASRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC